SEQUENCE LISTING (104 sequences)

SEQ ID NO: 1 - DNA, 20 bp, Artificial Sequence
  PAL1F
gtgtcgcacttcagaaggaa

SEQ ID NO: 2 - DNA, 20 bp, Artificial Sequence
  PAL1R
ggcttgtttctttcgtgctt

SEQ ID NO: 3 - DNA, 20 bp, Artificial Sequence
  PAL2F
gtgctacttctcaccggaga

SEQ ID NO: 4 - DNA, 20 bp, Artificial Sequence
  PAL2R
tattccggcgttcaaaaatc

SEQ ID NO: 5 - DNA, 18 bp, Artificial Sequence
  PAL3F
caaccaaacgcaacagca

SEQ ID NO: 6 - DNA, 20 bp, Artificial Sequence
  PAL3R
ctccaggtggctccctttta

SEQ ID NO: 7 - DNA, 20 bp, Artificial Sequence
  PAL4F
ggtgcacttcaaaatgagct

SEQ ID NO: 8 - DNA, 19 bp, Artificial Sequence
  PAL4R
caacgtgtgtgacgtgtcc

SEQ ID NO: 9 - DNA, 21 bp, Artificial Sequence
  C4HF
tgagtttggatccagaacgag

SEQ ID NO: 10 - DNA, 24 bp, Artificial Sequence
  C4HR
cgtcatgattcttctcatcttcct

SEQ ID NO: 11 - DNA, 21 bp, Artificial Sequence
  CHSF
cgcatcaccaacagtgaacac

SEQ ID NO: 12 - DNA, 20 bp, Artificial Sequence
  CHSR
tcctccgtcagatgcatgtg

SEQ ID NO: 13 - DNA, 20 bp, Artificial Sequence
  CHIF
ccggttcatcgatcctcttc

SEQ ID NO: 14 - DNA, 23 bp, Artificial Sequence
  CHIR
atcccggtttcagggatactatc

SEQ ID NO: 15 - DNA, 22 bp, Artificial Sequence
  F3HF
cagatcgttgaggcttgtgaga

SEQ ID NO: 16 - DNA, 23 bp, Artificial Sequence
  F3HR
acgagtcatatccgccactaagt

SEQ ID NO: 17 - DNA, 20 bp, Artificial Sequence
  F3′HF
gctctcgccggagtattcaa

SEQ ID NO: 18 - DNA, 20 bp, Artificial Sequence
  F3′HR
ccagcgacgccttgtaaatc

SEQ ID NO: 19 - DNA, 20 bp, Artificial Sequence
  DFRF
aacggatgtgacggtgtttt

SEQ ID NO: 20 - DNA, 20 bp, Artificial Sequence
  DFRR
tccattcactgtcggcttta

SEQ ID NO: 21 - DNA, 21 bp, Artificial Sequence
  LDOXF
cgatgaaaagatccgtgagaa

SEQ ID NO: 22 - DNA, 20 bp, Artificial Sequence
  LDOXR
cactccccaatccaaagatg

SEQ ID NO: 23 - DNA, 20 bp, Artificial Sequence
  FLSF
ccgtcgtcgatctaagcgat

SEQ ID NO: 24 - DNA, 17 bp, Artificial Sequence
  FLSR
cgtcggaatcccgtggt

SEQ ID NO: 25 - DNA, 22 bp, Artificial Sequence
  UDP-GSTF
gatcagaggaagtgatcgagga

SEQ ID NO: 26 - DNA, 21 bp, Artificial Sequence
  UDP-GSTR
gccaaaacagctgtctgagaa

SEQ ID NO: 27 - DNA, 21 bp, Artificial Sequence
  UGT78D3F
ctcctccgatatccccacaaa

SEQ ID NO: 28 - DNA, 21 bp, Artificial Sequence
  UGT78DSR
tcaacacgaatccctcaggaa

SEQ ID NO: 29 - DNA, 18 bp, Artificial Sequence
  GSTF
cttccgcaacccttttgg

SEQ ID NO: 30 - DNA, 17 bp, Artificial Sequence
  GSTR
ggctatgcccgcaatgt

SEQ ID NO: 31 - DNA, 23 bp, Artificial Sequence
  UF3GTF
caactggttttccgtttctggtt

SEQ ID NO: 32 - DNA, 22 bp, Artificial Sequence
  UF3GTR
gcttcctcgacggttgatacac

SEQ ID NO: 33 - DNA, 20 bp, Artificial Sequence
  Acylation F
cttccctggagctggaatct

SEQ ID NO: 34 - DNA, 17 bp, Artificial Sequence
  Acylation R
gccgctggatttggtca

SEQ ID NO: 35 - DNA, 22 bp, Artificial Sequence
  Acylation F
cagagccacttttacattgagc

SEQ ID NO: 36 - DNA, 21 bp, Artificial Sequence
  Acylation R
tcatccttgtcttcctcgttg

SEQ ID NO: 37 - DNA, 20 bp, Artificial Sequence
  Acylation F
agccacgctcctccactatc

SEQ ID NO: 38 - DNA, 20 bp, Artificial Sequence
  Acylation R
acggcatctttgtcgtcagg

SEQ ID NO: 39 - DNA, 25 bp, Artificial Sequence
  SAT F
ctgctgctatagtcaagtctcttcc

SEQ ID NO: 40 - DNA, 23 bp, Artificial Sequence
  SAT R
gagagaagaacatccaggtcctc

SEQ ID NO: 41 - DNA, 19 bp, Artificial Sequence
  RHM1F
ggtgggcgacactttgatg

SEQ ID NO: 42 - DNA, 25 bp, Artificial Sequence
  RHM1R
catatgggtttgacttggtttttca

SEQ ID NO: 43 - DNA, 23 bp, Artificial Sequence
  RHM2F
tctacaatgtcggcacaaaaaga

SEQ ID NO: 44 - DNA, 21 bp, Artificial Sequence
  RHM2R
ttcccgaaaagtttgcagatg

SEQ ID NO: 45 - DNA, 25 bp, Artificial Sequence
  RHM3F
atgcagatggtaatcagacattcac

SEQ ID NO: 46 - DNA, 22 bp, Artificial Sequence
  RHM3R
aggatcttttgtctccggaaca

SEQ ID NO: 47 - DNA, 20 bp, Artificial Sequence
  PAP1F
aaatggcaccaagttcctgt

SEQ ID NO: 48 - DNA, 26 bp, Artificial Sequence
  PAP1R
tcagagctaagttttcctctcttgat

SEQ ID NO: 49 - DNA, 25 bp, Artificial Sequence
  PAP2F
gactgctgaagaagatagtctcttg

SEQ ID NO: 50 - DNA, 25 bp, Artificial Sequence
  PAP2R
gcccagctctcaaaggaacttgatg

SEQ ID NO: 51 - DNA, 21 bp, Artificial Sequence
  MYB11F
ggcgattgtaacccaagcatt

SEQ ID NO: 52 - DNA, 21 bp, Artificial Sequence
  MYB11R
tcacatgaggacacgtggaca

SEQ ID NO: 53 - DNA, 22 bp, Artificial Sequence
  MYB12F
tgatggggagttgcataacata

SEQ ID NO: 54 - DNA, 19 bp, Artificial Sequence
  MYB12R
aacgactccaccgatggac

SEQ ID NO: 55 - DNA, 26 bp, Artificial Sequence
  MYB111F
aataacaagaccaagaagaagaagaa

SEQ ID NO: 56 - DNA, 20 bp, Artificial Sequence
  MYB111R
agaaacattgtgaggccgtc

SEQ ID NO: 57 - DNA, 23 bp, Artificial Sequence
  MYB113F
atcttgttcttcgccttcataaa

SEQ ID NO: 58 - DNA, 22 bp, Artificial Sequence
  MYB113R
gcatcgttcatcgtgcttctta

SEQ ID NO: 59 - DNA, 23 bp, Artificial Sequence
  MYB114F
gtctcttgaggcagtgtattggt

SEQ ID NO: 60 - DNA, 20 bp, Artificial Sequence
  MYB114R
ttttcctgcaccgatttagc

SEQ ID NO: 61 - DNA, 24 bp, Artificial Sequence
  GL3F
agtgtttagccgttctcttctagc

SEQ ID NO: 62 - DNA, 24 bp, Artificial Sequence
  GL3R
tgtcttccgtaatatgttctgtgg

SEQ ID NO: 63 - DNA, 18 bp, Artificial Sequence
  EGL3F
ttggcacgaccgaacata

SEQ ID NO: 64 - DNA, 27 bp, Artificial Sequence
  EGL3R
ttgatagtctgatcttgtcgatattgt

SEQ ID NO: 65 - DNA, 23 bp, Artificial Sequence
  TT8F
tgaatcaacccatacgttagaca

SEQ ID NO: 66 - DNA, 21 bp, Artificial Sequence
  TT8R
ggggtgtgacatgagaagtgt

SEQ ID NO: 67 - DNA, 22 bp, Artificial Sequence
  TTG1F
tcctcgaagattacaacaaccg

SEQ ID NO: 68 - DNA, 21 bp, Artificial Sequence
  TTG1R
cgggagaggcttaacggtcat

SEQ ID NO: 69 - DNA, 20 bp, Artificial Sequence
  4CL1F
tcaacccggtgagatttgta

SEQ ID NO: 70 - DNA, 20 bp, Artificial Sequence
  4CL1R
tcgtcatcgatcaatccaat

SEQ ID NO: 71 - DNA, 20 bp, Artificial Sequence
  HCTF
gcctgcaccaagtatgaaga

SEQ ID NO: 72 - DNA, 20 bp, Artificial Sequence
  HCTR
gacagtgttcccatcctcct

SEQ ID NO: 73 - DNA, 20 bp, Artificial Sequence
  C3′H F
gttggacttgaccggatctt

SEQ ID NO: 74 - DNA, 20 bp, Artificial Sequence
  C3′HR
attagaggcgttggaggatg

SEQ ID NO: 75 - DNA, 20 bp, Artificial Sequence
  CCoAOMTF
ctcagggaagtgacagcaaa

SEQ ID NO: 76 - DNA, 20 bp, Artificial Sequence
  CCoAOMTR
gtggcgagaagagagtagcc

SEQ ID NO: 77 - DNA, 20 bp, Artificial Sequence
  CCR1F
gtgcaaagcagatcttcagg

SEQ ID NO: 78 - DNA, 20 bp, Artificial Sequence
  CCR1R
gccgcagcattaattacaaa

SEQ ID NO: 79 - DNA, 20 bp, Artificial Sequence
  F5HF
cttcaacgtagcggatttca

SEQ ID NO: 80 - DNA, 20 bp, Artificial Sequence
  F5HR
agatcattacgggccttcac

SEQ ID NO: 81 - DNA, 20 bp, Artificial Sequence
  COMT1F
ttccattgctgctctttgtc

SEQ ID NO: 82 - DNA, 20 bp, Artificial Sequence
  COMT1R
catggtgattgtggaatggt

SEQ ID NO: 83 - DNA, 26 bp, Artificial Sequence
  CAD3F
atcgtttcggatattgagctcataaa

SEQ ID NO: 84 - DNA, 26 bp, Artificial Sequence
  CAD3R
tctcagctgacgactcagggagtaaa

SEQ ID NO: 85 - DNA, 22 bp, Artificial Sequence
  CAD4F
tctggtggaggaggctgcaaca

SEQ ID NO: 86 - DNA, 25 bp, Artificial Sequence
  CAD4R
agccaaagcattcgtgtttgaacca

SEQ ID NO: 87 - DNA, 20 bp, Artificial Sequence
  CAD5F
ttggctgattcgttggatta

SEQ ID NO: 88 - DNA, 20 bp, Artificial Sequence
  CAD5R
atcactttcctcccaagcat

SEQ ID NO: 89 - DNA, 27 bp, Artificial Sequence
  CAD7F
cgatgaagccaactccttaactagaaa

SEQ ID NO: 90 - DNA, 31 bp, Artificial Sequence
  CAD7R
caactgataaagtacatgcagtgtggtaata

SEQ ID NO: 91 - DNA, 33 bp, Artificial Sequence
  CAD8F
cattgaagcctaatcctaatttataagttttaa

SEQ ID NO: 92 - DNA, 34 bp, Artificial Sequence
  CAD8R
actattcatttattggattaagcataccaaatta

SEQ ID NO: 93 - DNA, 27 bp, Artificial Sequence
  CAD9F
cctccatgaatgatccggatctaagaa

SEQ ID NO: 94 - DNA, 29 bp, Artificial Sequence
  CAD9R
cgaaataaggtaacttgtctgagaagaaa

SEQ ID NO: 95 - DNA, 24 bp, Artificial Sequence
  LAC4F
ggtggatgggtcgtcatgagattc

SEQ ID NO: 96 - DNA, 26 bp, Artificial Sequence
  LAC4R
cgtggcgtgatgttgatatgtcgccc

SEQ ID NO: 97 - DNA, 21 bp, Artificial Sequence
  LAC17F
ggccatttatcggtttgacat

SEQ ID NO: 98 - DNA, 21 bp, Artificial Sequence
  LAC17R
cagaaaacagagctgtgcaac

SEQ ID NO: 99 - DNA, 21 bp, Artificial Sequence
  SND1F
cccgagttcgctttccaggtg

SEQ ID NO: 100 - DNA, 21 bp, Artificial Sequence
  SND1R
caggacgaaccgggcaacagt

SEQ ID NO: 101 - DNA, 24 bp, Artificial Sequence
  MYB58F
ccagagaacagagctcttcaagag

SEQ ID NO: 102 - DNA, 24 bp, Artificial Sequence
  MYB58R
atgtatgaggagctcgtaactctc

SEQ ID NO: 103 - DNA, 24 bp, Artificial Sequence
  MYB63F
gaacagctcaggctcaagagcaac

SEQ ID NO: 104 - DNA, 24 bp, Artificial Sequence
  MYB63R
atgtatcatgagctcgtagttctt

(19) United States
(12) Patent Application Publication
(10) Pub. No.: US 2014/0073504 A1
(43) Pub. Date: Mar. 13, 2014

(54) USE OF MICROBIAL BASED SOIL ADDITIVE TO MODULATE THE PHENYLPROPANOID PATHWAY IN PLANTS

(71) Applicants: University of Kentucky, Lexington, KY (US); Tenfold Technologies, LLC, Pilot Point, TX (US)

(72) Inventors: David McNear, Lexington, KY (US); Mohammed B Ali, Lexington, KY (US); Robert Norman Ames, Pilot Point, TX (US)

(21) Appl. No.: 13/832,110

(22) Filed: Mar. 15, 2013

Related U.S. Application Data
(60) Provisional application No. 61/673,696, filed on Jul. 19, 2012.

(51) Int. Cl. A01N 63/00; C12Q 1/68

(52) U.S. Cl. CPC A01N 63/00; C12Q 1/6876
USPC 504/117; 435/6.11; 435/6.12; 536/24.3; 536/24.33

(57) ABSTRACT

Provided is a method for inducing, stimulating and/or otherwise promoting and/or modulating flavonoid and/or lignin biosynthesis in a plant by using a microbial based soil additive or amendment.

### TECHNICAL FIELD

Provided herein is the use of microbial inoculants to influence plant secondary metabolism, particularly the phenylpropanoid pathway. Such use may act to improve the quality of agricultural products, in terms of nutritional content and flavor, as substrate for biofuels and biomaterials, and/or to improve plant's defense against pathogens and pests.

### BACKGROUND

One of the major challenges in the 21^{st} century is the sustainable production of food, fuel and fiber crops with enhanced functional and nutritive value (e.g. flavonoids and anthocyanidins) to meet the demands of an ever-increasing global population (Green et al., 2005; Vandermeer and Perfecto, 2005; DeFries and Rosenzweig, 2010). The development of alternative more sustainable methods for the production and enhancement of value added agricultural commodities in a way that will have minimal impact on the ecosystem is required to meet this demand. Current agricultural practices are largely based on the use of chemical fertilizers and synthetic pesticides for improved crop growth and yield. However, dependence and overuse of these fertilizers has resulted in contamination of soil, ground and surface waters. Increasing demand for healthier and more nutrient-dense foods by more health-conscious consumers and an improved environmental awareness has resulted in an increased interest in and a rapid change towards eco-friendly sustainable agricultural farming systems.

One component of this new sustainable production system is the use of microbe-based fertilizer amendments (i.e. biostimulants) containing potential beneficial strains of microorganisms and their metabolites many of which have an important role in conditioning the rhizosphere for improved plant growth and nutrient use efficiency (Saber, 2001; Whipps, 2001; Barea et al., 2005; Morgan et al., 2005). There have been many reports on improvements in plant defense, health and growth, resistance to pathogens, enhanced salt tolerance, and improved nutrient uptake in response to plant growth promoting rhizobacteria (PGPR) (Walsh et al., 2001; Lugtenberg et al., 2002; Adesemoye et al., 2009; Morrissey et al., 2004; Dodd and Perez-Alfocea, 2012) that could have led to the development of novel agricultural applications. In spite of all these advantages, the use of microbial-based products has not been effectively exploited at larger scales to improve plant yields and most certainly not as a means to selectively enhance gene expression and production of beneficial secondary metabolite in crops.

Phenylpropanoids are a large group of polyphenolic compounds that comprise an important class of secondary metabolites such as flavonoids, anthocyanin and lignin in plants (Ververidis et al., 2007). Phenylpropanoids have important functions in flower coloration, pollinator attraction, protection from ultraviolet (UV) light, as signaling molecules between plants and microbes, and as antioxidants (Kutchan, 2005). Additionally, when consumed by humans phenylpropanoids offer a myriad of health benefits (García-Mediavilla et al., 2007; Sung et al., 2011). There have been many studies on the biosynthesis of flavonols and the phenylpropanoid (PP) pathway in general via metabolic engineering targeting important agronomic traits such as the production of novel colors and color patterns in ornamentals (Ververidis et al., 2007; Ruiz-Lopez et al., 2012). The synthesis of metabolites such as flavonoids and anthocyanin is governed by several structural genes and regulatory genes of several families. The phenylpropanoid pathway is set forth in FIG. 1. Transcription factors (MYB75, 90, 11, 12, 111, EGL3 and GL3) and structural genes (PALs, CHS, CH1, F3H, F3′H, FLS1, DFR, LDOX and UF3GT) play important role in the biosynthesis of flavonoids in Arabidopsis. Myb75, Myb90, EGL3 and GL3 regulate the flavonoids biosynthesis. Myb 11, 111 and 112 have been demonstrated to be regulators for the ‘early’ gene expression (CHS, CH1, F3H and FLS1) (Mehrtens et al., 2005; Stracke et al., 2007).

### SUMMARY

Provided is a method for inducing, stimulating or otherwise promoting and/or modulating at least one of: (a) flavonoid biosynthesis; and (b) lignin biosynthesis in a plant in need thereof comprising applying to soil and/or said plant an amount of a microbial based soil additive and/or amendment effective to induce and/or modulate flavonoid and/or lignin biosynthesis. This method may be used with a monocotyledonous or dicotyledenous plants and may include but is not limited to plant crops such as fruit (e.g., strawberry), vegetable (e.g., tomato, squash, pepper, eggplant), grain crops (e.g., soy, wheat, rice, corn), fiber crops, (e.g., cotton), tree, flower, ornamental plant, shrub (e.g. rose), bulb plant (e.g, onion, garlic) or vine (e.g., grape vine).

Also provided is a method for detecting induction of flavonoid biosynthesis in a plant in need thereof by a microbial based soil additive comprising:

(a) providing a sample from a plant and

(b) contacting said sample with one or more probe or primers having at least about 97% homology to a nucleotide sequence wherein said nucleotide sequence is SEQ ID NOS; 1-38 and 41-68. In a particular embodiment, the method further comprises comparing the level of flavonoid biosynthesis to the level of flavonoid biosynthesis in a sample from a plant not treated. In a related aspect, provided is an oligonucleotide probe or primer for detecting flavonoid biosynthesis in a plant having a sequence of at least 17 nucleotides with at least about 97% homology to a nucleotide sequence, wherein said sequence is selected from the group consisting of SEQ ID NOS: 1-38 and 41-68.

In a similar aspect, a method is provided for detecting induction of lignin biosynthesis in a plant in need thereof by a microbial based soil additive comprising:

(a) providing a sample from a plant and

(b) contacting said sample with one or more probe or primers having at least about 97% homology to a nucleotide sequence wherein said nucleotide sequence is SEQ ID NOS:39-40 and 70-103. In a particular embodiment, the method further comprises comparing the level of flavonoid biosynthesis to the level of flavonoid biosynthesis in a sample from a plant not treated.

Further provided are oligonucleotides that may be used to detect induction of flavonoid biosynthesis. In a particular embodiment, the oligonucleotide has at least about 97% homology to SEQ ID NO: 9, 10, 33, 34, 47, 48, 53, 54, 55, 56, 67, 68. These oligonucleotides are at least about 17 nucleotides in length and in a particular embodiment may have a length of about 20 nucleotides, 25 nucleotides, 30 nucleotides or 35 nucleotides.

Further provided are oligonucleotides that may be used to detect induction of lignin biosynthesis. In a particular embodiment, the oligonucleotide has at least about 97% homology to SEQ ID NO: 39-40. These oligonucleotides are at least about 17 nucleotides in length and in a particular embodiment may have a length of about 20 nucleotides, 25 nucleotides, 30 nucleotides or 35 nucleotides.

Also provided is a kit for detecting flavonoid and/or lignin biosynthesis in a plant comprising SEQ ID NOS: 1-104.

### BRIEF DESCRIPTION OF THE FIGURES

FIG. 1 provides an overview of phenylpropanoid and lignin biosynthesis pathway in Arabidopsis.

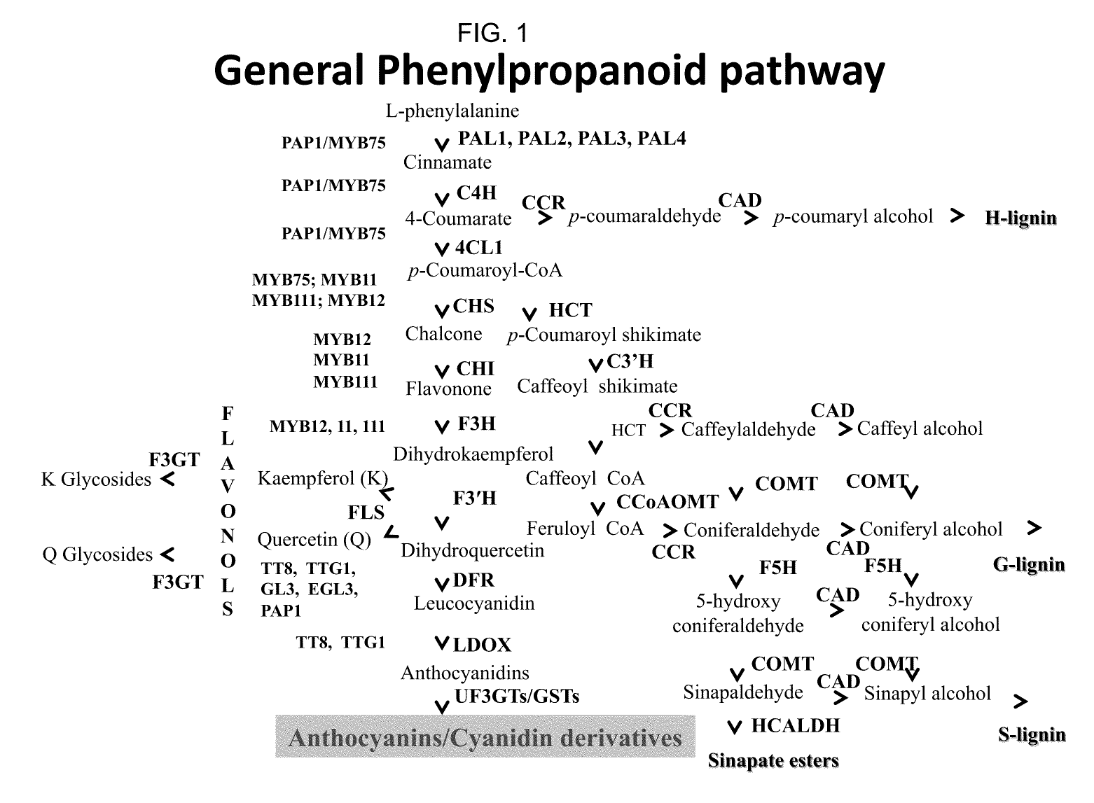

FIG. 2 shows profiles of flavonol glycoside in Arabidopsis thaliana treated once (TI) and multiple times (TII) with SoilBuilder™-AF (Agricen, Pilot Point, Tex.). Kaempferol-3,7-O-bis-alpha-L-rhamnoside (F1), kaempferol-3-O-alpha-L-rhamnopyranosyl (1-2)-beta-D-glucopyranoside-7-O-alpha-L-rhamnopyranoside (F2), Kaempferol with rhamnoside at Rt 7.3 (F3), Kaempferol with rhamnoside at Rt 8.4 (F4), Kaempferol with rhamnoside at Rt 7.8 (F5), Kaempferol in hydrolyzed at Rt 11.4 (F6), quercetin 3,7-dirhamnoside (F7), apigenin 7-(2″,3″-diacetylglucoside) (F8) and pentamethoxydihydroxyflavone (F9).

FIG. 3 shows profiles of anthocyanidins glycoside in Arabidopsis thaliana treated once (TI) and multiple times (TII) with SoilBuilder™-AF (Agricen, Pilot Point, Tex.). Cyanidin-Rhamnoglucoside (A1), cyanidin 3-(6-malonylglucoside)-7,3′-di-(6-feruloylglucoside) (A2), cyanidin 3-(6″-caffeyl-2′″-sinapylsambubioside)-5-(6-malonylglucoside) (A3) and cyanidin 3-(2G-glucosylrutinoside) at Rt 7.1 (A4) and cyanidin 3-(2G-glucosylrutinoside) at Rt 7.6 (A5).

FIG. 4 shows relative transcript abundance of phenylalanine ammonia lyases (PAL1, PAL2, PAL3 and PAL4) genes known to be involved in flavonoid biosynthesis in Arabidopsis thaliana after being treated once (TI) and multiple times (TII) with SoilBuilder™ AF (Agricen, Pilot Point, Tex.). Primers used in these studies, products size for the amplified fragments, accession numbers are shown in Table 2. Transcript abundance of each gene was normalized by the level of an actin and EF-1α gene. Bars indicate standard error of three biological replicates at each sampling time-point.

FIG. 5 shows relative transcript abundance of structural genes (CHS, CHI, F3H, F3′H, FLS1, UF3GT, DFR and LDOX) known to be involved in flavonoid biosynthesis in Arabidopsis treated once (TI) and multiple times (TII) with SoilBuilder™-AF (Agricen, Pilot Point, Tex.). Primers used in these studies, products size for the amplified fragments, accession numbers are shown in Table 2. Transcript abundance of each gene was normalized by the level of an actin and EF-1α gene. Bars indicate standard error of three biological replicates at each sampling time-point. Different letters in different bar differ significantly from the control according to Fit Least Squares (FLS) test, P≦0.05. CONT (black bar) indicates the untreated plants, TI (shaded) and TII (white) treated with microbial products only once and multiple times, respectively.

FIG. 6 shows relative transcript abundance of acetyltransferase genes At1g03495, At1g03940, and At3g29590 known to be involved in the acylation (A) UDP-glucosyltransferase At4g14090 and At5g17030 and GSTs At1g02920 genes known to be involved in the glycosylation (B), and rhamnose synthesis genes (RHM1, RHM2 and RHM3) involved in the rhamnosylation (C) of flavonoids treated once (TI) and multiple times (TII) with SoilBuilder™-AF (Agricen, Pilot Point, Tex.) in Arabidopsis thaliana. Primers used in these studies, products size for the amplified fragments, accession numbers are shown in Table 2. Transcript abundance of each gene was normalized by the level of an actin and EF-1α gene. Bars indicate standard error of three biological replicates at each sampling time-point.

FIG. 7 shows relative transcript abundance of transcription factors (PAP1, PAP2, MYB11, MYB12, MYB111, MYB113, MYB114, GL3, EGL3, TT8 and TTG1) known to direct flavonoids biosynthesis-related gene expression in Arabidopsis treated once (TI) and multiple times (TII) with SoilBuilder™-AF (Agricen, Pilot Point, Tex.). Primers used in these studies, products size for the amplified fragments, accession numbers are shown in Table 2. Transcript abundance of each gene was normalized by the level of an actin and EF-1α gene. Bars indicate standard error of three biological replicates at each sampling time-point. Different letters in different bar differ significantly from the control according to Fit Least Squares (FLS) test, P≦0.05. CONT (black bar) indicates the untreated plants, TI (shaded) and TII (white) treated with microbial products only once and multiple times, respectively.

FIG. 8 shows relative transcript abundance of lignin pathway structural genes (C4H, 4CL1, HCT, C3′H, CCoAOMT, CCR1, CAD, COMT, F5H and SAT) known to be involved in lignin biosynthesis in Arabidopsis treated once (TI) and multiple times (TII) with SoilBuilder™-AF (Agricen, Pilot Point, Tex.). Primers used in these studies, products size for the amplified fragments, accession numbers are shown in Table 3. Transcript abundance of each gene was normalized by the level of an actin and EF-1α gene. Bars indicate standard error of three biological replicates at each sampling time-point. Different letters in different bar differ significantly from the control according to Fit Least Squares (FLS) test, P≦0.05. CONT (black bar) indicates the untreated plants, TI (shaded) and TII (white) treated with microbial products only once and multiple times, respectively.

FIG. 9 shows relative transcript abundance of cinnamyl alcohol dehydrogenase family genes (CAD1, CAD3, CAD4, CAD5, CAD7 and CAD8) known to be involved in lignin biosynthesis treated once (TI) and multiple times (TII) with SoilBuilder™-AF (Agricen, Pilot Point, Tex.) in Arabidopsis thaliana. Primers used in these studies, products size for the amplified fragments, accession numbers are shown in supplemental Table 2. Transcript abundance of each gene was normalized by the level of an actin and EF-1α gene. Bars indicate standard error of three biological replicates at each sampling time-point.

FIG. 10 shows relative transcript abundance of laccase (LAC4 and LAC17) genes known to be involved in lignin biosynthesis (A) and transcription factors (SND1, MYB58 and MYB63) (B) are known to regulate lignin biosynthesis in Arabidopsis treated once (TI) and multiple times (TII) with SoilBuilder™-AF in Arabidopsis thaliana. Primers used in these studies, products size for the amplified fragments, accession numbers are shown in supplemental Table 3. Transcript abundance of each gene was normalized by the level of an actin and EF-1α gene. Bars indicate standard error of three biological replicates at each sampling time-point.

FIG. 11 shows the influence of microbial products SoilBuilder™-AF (Agricen, Pilot Point, Tex.) treated once (TI) and multiple times (TII) on lignin content in Arabidopsis thaliana. Bars indicate standard error of three biological replicates at each sampling time-point. Different letters in different bar differ significantly from the control according to Fit Least Squares (FLS) test, P≦0.05. CONT (black bar) indicates the untreated plants, TI (shaded) and TII (white) treated with microbial products only once and multiple times, respectively.

### DETAILED DESCRIPTION

While the compositions and methods heretofore are susceptible to various modifications and alternative forms, exemplary embodiments will herein be described in detail. It should be understood, however, that there is no intent to limit the invention to the particular forms disclosed, but on the contrary, the intention is to cover all modifications, equivalents, and alternatives falling within the spirit and scope of the invention as defined by the appended claims.

### DEFINITIONS

Where a range of values is provided, it is understood that each intervening value, to the tenth of the unit of the lower limit unless the context clearly dictates otherwise, between the upper and lower limit of that range and any other stated or intervening value in that stated range, is included therein. Smaller ranges are also included. The upper and lower limits of these smaller ranges are also included therein, subject to any specifically excluded limit in the stated range.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

As defined herein, “derived from” means directly isolated or obtained from a particular source or alternatively having identifying characteristics of a substance or organism isolated or obtained from a particular source.

As defined herein “modulate” means adjusting amount and/or rate of flavonoid biosynthesis or lignin biosynthesis.

Plants “in need thereof” are plants that are being cultivated and need to have their growth rate, amount of growth and/or host defense mechanism boosted.

The terms “polynucleotide(s)”, “nucleic acid molecule(s)” and “nucleic acids” will be used interchangeably.

The terms “percent homology”, “percent similarity” and “percent identity” are used interchangeably.

“Percent Identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base occurs in both sequences to yield the number of matched positions, dividing the number of matched positions by the total number of positions in the window of comparison, and multiplying the result by 100 to yield the percentage of sequence identity.

Optimal alignment of sequences for comparison can use any means to analyze sequence identity (homology) known in the art, e.g., by the progressive alignment method of termed “PILEUP” (Morrison, 1997), as an example of the use of PILEUP); by the local homology algorithm of Smith & Waterman, (1981); by the homology alignment algorithm of Needleman & Wunsch (1970); by the search for similarity method of Pearson (1988); by computerized implementations of these algorithms (e.g., GAP, BEST FIT, FASTA, and TFASTA in the Wisconsin Genetics Software Package, Genetics Computer Group, 575 Science Dr., Madison, Wis.); ClustalW (CLUSTAL in the PC/Gene program by Intelligenetics, Mountain View, Calif., described by, e.g., Higgins (1988); Corpet (1988); Huang (1992); and Pearson (1994); Pfamand Sonnhammer (1998); TreeAlign (Hein (1994); MEG-ALIGN, and SAM sequence alignment computer programs; or, by manual visual inspection.

Another example of an algorithm that is suitable for determining sequence similarity is the BLAST algorithm, which is described in Altschul et al., (1990). The BLAST programs (Basic Local Alignment Search Tool) of Altschul, S. F., et al., (1993) searches under default parameters for identity to sequences contained in the BLAST “GENEMBL” database. A sequence can be analyzed for identity to all publicly available DNA sequences contained in the GENEMBL database using the BLASTN algorithm under the default parameters. Software for performing BLAST analyses is publicly available through the National Center for Biotechnology Information, www.ncbi.nlm.nih.gov/; see also Zhang (1997) for the “PowerBLAST” variation. This algorithm involves first identifying high scoring sequence pairs (HSPs) by identifying short words of length W in the query sequence that either match or satisfy some positive valued threshold score T when aligned with a word of the same length in a database sequence. T is referred to as the neighborhood word score threshold (Altschul et al., (1990)). These initial neighborhood word hits act as seeds for initiating searches to find longer HSPs containing them. The word hits are extended in both directions along each sequence for as far as the cumulative alignment score can be increased. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a wordlength (W) of 11, the BLOSUM62 scoring matrix (see Henikoff (1992)) alignments (B) of 50, expectation (E) of 10, M=5, N=−4, and a comparison of both strands. The term BLAST refers to the BLAST algorithm which performs a statistical analysis of the similarity between two sequences; see, e.g., Karlin (1993).

Yet another example of an algorithm is the GenEx program (MultiD) that provides methods for analyzing real time qPCR data of individual genes by specifically providing means for comparing test sequences to reference genes. The GenEx program is particularly useful for generating quantitiative data.

### LIST OF ABBREVIATIONS

C3′H: p-coumarate 3-hydroxylase; PAL: phenylalanine ammonia-lyase; CHS, chalcone synthase; CHI, chalconeisomerase; UF3GT, UDP-glucose:flavonoid-3-O-glucosyltransferase; F3H, flavanone 3-hydroxylase; F3′H, flavonoid-3′-O-hydroxylase; DFR, dihydroflavonol-4-reductase; LDOX, leucoanthocyanidindioxygenase; UDP-GST: UDP-glucoronosyl/UDP-glucosyltransferase; GST, glutathione S-transferase; FLS1: flavonol synthase1; PAP1 & 2: production of anthocyanin pigment1 & 2; EGL3: enhancer of glabra3; GL3: glabrous 3; 3GlcT: flavonoid 3-O-glucosyltransferase; 3RhaT: flavonol 3-O-rhamnosyltransferase; 7GlcT:flavonol 7-O-glucosyltransferase; 5GlcT: anthocyanin 5-O-glucosyltransferase; PGPR: plant growth-promoting rhizobacteria.

### Description of Specific Embodiments

Method of Modulating Flavonoid and/or Lignin Biosynthesis

As noted above, provided is a method for inducing, stimulating or otherwise promoting and/or modulating at least one of: (a) flavonoid biosynthesis; and (b) lignin biosynthesis. In a particular embodiment, biosynthesis of at least one of the flavonoids is induced: Anthocyanidin 3-(2G-glucosylrutinoside), Kaempferol-3-O-alpha-L-rhamnopyranosyl(1-2)-beta-D-glucopyranoside-7-O-alpha-L-rhamnopyranoside, Kaempferol with rhamnosides, Anthocyanidin 3-Rhamnoglucoside, Anthocyanidin 3-(2G-glucosylrutinoside), Kaempferol with rhamnosides, Quercetin 3,7-dirhamnoside, Anthocyanidin 3-(6-malonylglucoside)-7,3′-di-(6-feruloylglucoside), Anthocyanidin 3-(6″-caffeyl-2′″-sinapylsambubioside)-5-(6-malonylglucoside), Pentamethoxydihroxyflavone, Apigenin 7-(2″,3″-diacetylglucoside), Kaempferol-3,7-O-bis-alpha-L-rhamnoside, Kaempferol.

In one embodiment, the method, flavonoid biosynthesis and/or lignin biosynthesis may be induced, stimulated or otherwise promoted and/or modulated by inducing, stimulating or otherwise promoting and/or modulating expression of (a) one or more structural genes and/or (b) one or more glycosylation gene, and/or (c) one or more acylation genes and/or (d) one or more regulatory genes of the phenylpropanoid pathway by applying to soil and/or said plant an amount of a soil additive derived from a bioreactor effective to induce and/or modulate expression of structural genes, and/or regulatory genes of the phenylpropanoid pathway. The structural gene induced, stimulated or otherwise promoted and/or modulated includes but is not limited to (a) one or more isoforms of PAL (e.g., PAL1, PAL2, PAL3, PAL4), (b) F3H, (c) F3′H, (d) FLS1, (e) DFR, (f) LDOX, (g) C4H; (h) 4CL1, (i) CHS, (j) CH1 and/or (j) UF3GT.

Glycoslyation may be induced, stimulated or otherwise promoted and/or modulated by, for example, inducing, stimulating or otherwise promoting and/or modulating the expression of a UDP glycosyl transferase gene (e.g., UGT75C1, UGT78D3, UDP) rhamnose synthase gene (RHM1, RHM2 and RHM3) or glutathione-S-transferase gene (e.g., GST). The expression of the acylation gene induced, stimulated or otherwise promoted and/or modulated may be At1g03495, At1g03940 and/or At3g29590 gene. The expression of the regulatory gene induced, stimulated or otherwise promoted and/or modulated may, for example, be one or more of a MYB (e.g., MYB11, MYB12, MYB113, MYB114, EGL3, GL3, TT8 and/or TTG1) and/or PAP transcription factor (e.g., PAP1 and/or PAP2).

In yet another particular embodiment, flavonoid biosysnthesis may be induced, stimulated or otherwise promoted and/or modulated by inducing, stimulating or otherwise promoting and/or modulating at least one of methylation, acylation and/or glycosylation of a flavonoid in a plant in need thereof comprising applying to soil and/or said plant an amount of a microbial based soil additive effective to induce and/or modulate methylation, acylation and/or glycosylation of said flavonoid. Methods for methylating, acylating and/or glycosylating said flavonoid may be performed using methods known in the art and also using methods set forth above for acylation and glycosylation. Methylation may also be performed with a glucosyltransferase. In yet another particular embodiment, the flavonoid may be kaempferol or derivative thereof, an apigenin or derivative thereof, a quercitin or derivative thereof, a dihydroxyflavone and/or cyanadin derivative. In a more specific embodiment, the kaempferol derivative, cyanidin derivative and/or quercetin derivative is a glycosylated derivative. In yet another specific embodiment, the cyanidin derivative is an acylated derivative. In yet another specific embodiment, a dihydroxyflavone derivative is methylated.

Lignin biosynthesis, in another particular embodiment, may be induced, stimulated or otherwise promoted and/or modulated by applying an amount of a microbial based soil additive effective to induce, stimulate or otherwise promote and/or modulate expression of at least one of (a) one or more lignin biosynthesis genes; (b) one or more laccase genes and/or (c) one or more or more lignin regulatory genes. In a particular embodiment, the lignin biosynthesis gene includes but is not limited to 4CL1, HCT, C3′H1, CCoAOMT, CCR1, CCR2, COMT1, CAD1, CAD3, CAD5, CAD7, CAD8 and SAT. In another particular embodiment, the laccase gene induced, stimulated or otherwise promoted and/or modulated includes but is not limited to LAC4 and LAC17. In yet another particular embodiment, the lignin regulatory genes induced, stimulated or otherwise promoted and/or modulated includes, but is not limited to, MYB63, C3′HY63, SND1 and MYB58.

The microbial based soil additive or amendment used in these methods may be derived from microbial based material that adds nutrients such as carbon and nitrogen, as well as beneficial bacteria to soil and when applied to soil improve its physical properties, such as water retention, permeability, water infiltration, drainage, aeration and structure. In a particular embodiment, the microbial based soil additive or amendment may be derived from a microbial consortium comprising a consortium of microbial (e.g. bacterial) species. This microbial consortium may be derived from feedstock processed through a bioreactor. It would be beneficial to apply said microbial composition as a seed-treatment and/or to the plant at the seedling stage.

In a particular embodiment, the soil additive and/or amendment is a composition that has the following characteristics: (a) has a pH of about 7.5 to 8.8; (b) COD range less than about 150 mg/L; (c) Conductivity range of about 1200 uS to 3800 uS; (d) Color clear amber between about 500 pt/co units to about 700 pt/co units in a platinum to cobalt (pt/co) scale; (e) comprises at least one of Syntrophus, Desulfovibrio, Symbiobacteria, Georgfuschia, Thauera, Nitrosomonas, Bellilinea, Sulfuritalea, and Owenweeksia; (f) has a culturable plate count of greater than 10⁶ microbes per ml.; (g) contains between about 10-60 ng/ml DNA; (h) comprises at least eight microbial species or filter-sterilized broth thereof.

In a particular embodiment the microbial based soil additive and/or amendment is set forth in PCT/US 2012/060010. This microbial product contains microbes and microbially-produced metabolites.

In a more particular embodiment, the microbial based soil additive is derived from SoilBuilder™-AF (Agricen, Pilot Point, Tex.) (SB) and even more particularly from a filter-sterilized broth of SoilBBuilder™-AF (Agricen, Pilot Point, Tex.) (FSB). SoilBuilder, a commercially available microbial soil amendment is prepared from a bioreactor system consisting of a continuously maintained microbial community. The final product contains bacteria and bacterial metabolites derived from the bioreactor. Based on plate counts using tryptic soy agar (TSA) (incubation for 24 h at 25 C). the most commonly occurring bacteria within the I111a1 stabilized product are Acidovoras bacillus, Bacillus licheniformis, Bacillus subtilis, Bacillus oleronius, Bacillus marinus, Bacillus megaterium, and Rhodococcus rhodochrous, each at 1×10³ colony-forming units (cfu) mL⁻¹.

Detection of Induction of Flavonoid and/or Lignin Biosynthesis

The induction of flavonoid and/or lignin biosynthesis in a plant can be detected by a number of methods. In all of these methods, an extract derived from a plant part is obtained. The extract may as set forth in the Example below may be subjected to detection methods including but not limited to mass spectroscopy to identify and quantify flavonoids and lignin

Alternatively, nucleic acid may be obtained from a plant or plant part (e.g., leaf, stem, roots, flower using methods known in the art. A nucleic acid (e.g., DNA or RNA) may be obtained from a sample from a plant or plant part (e.g., leaf, stem, roots, flower), using methods known in the art (e.g., nucleic acid extraction). This nucleic acid may be hybridized to a probe or primer using methods known in the art.

Alternatively, the probe or primer may act as primer for amplification of a sequence or synthesis of a cDNA or cRNA sequence by, for example, PCR reaction. In a particular embodiment, the probe may comprise a nucleotide sequence having at least about 97% identity to SEQ ID NOS:1-104. In more particular embodiments, the probe or primer comprises a nucleotide sequence that has greater than about 97%, 98%, 99%, or 99.5% identity to SEQ ID NOS: 1-104. In an even more particular embodiment, the probe or primer comprises a nucleotide sequence that has greater than about 97%, 98%, 99%, or 99.5% identity to SEQ ID NOS: SEQ ID NO: 9, 10, 33, 34, 39, 40, 47, 48, 53, 54, 55, 56, 67, 68. The probes or primers are at least 17 nucleotides in length and may range from about 17 nucleotides in length to about 25 nucleotides.

The PCR reaction products in the sample may be compared with various flavonoid and/or lignin regulatory genes sequences using various methods known in the art, including but not limited to BLAST and NCBI,

The probes or primers used may be packaged into test kits. These kits may further contain detectable labels and written instructions. In a particular embodiment, the probes or primers may be attached to solid supports.

### Example

The composition and methods set forth above will be further illustrated in the following, non-limiting Examples. The examples are illustrative of various embodiments only and do not limit the claimed invention regarding the materials, conditions, weight ratios, process parameters and the like recited herein.

### Materials and Methods

### Source of Microbial Preparation

SoilBuilder™-AF (Agricen, Pilot Point, Tex., USA), a biochemical fertilizer catalyst developed specifically for the agriculture industry, contains bacterial products derived from a bioreactor system consisting of a large and diverse microbial community. The microbial community composition of SoilBuilder™-AF (Agricen, Pilot Point, Tex., USA) has been assessed using 16S rRNA based gene analysis and is generally composed of species of, in addition to the above mentioned bacterial species: Bacillus, Actimomyces including Proteobacteria. Previous works also reported that SoilBuilder consists of Bacillus species, Actimomyces, Cyanobacteria, algae, protozoa, and microbial by-products (Yildirim et al., 2006) including microbial metabolites produced during anaerobic fermentation of a microbial community (Burkett-Cadena et al., 2008). Basic chemical composition of the product was determined by the University of Kentucky Soil Testing Laboratory following standard protocols.

### Growth Conditions and Treatment Procedure

Seeds of Arabidopsis thaliana ecotype Columbia-0 were sterilized and sown on solid 0.7% agar plates containing 1× Murashige and Skoog medium (pH 5.7). Plates were incubated in darkness at 4° C. for 2-3 days and were transferred to a growth chamber at 22° C. with a 16-h light/8-h dark cycle at a photosynthetic photon flux density (PPFD) of 100 μmol m⁻² s⁻¹, and 65-70% relative humidity and grown for two weeks. After two weeks seventy of the seedlings were transferred to six inch pots containing fertilizer (PRO-MIX® BX, Quakertown, Pa., USA), arranged in randomized complete block design in the growth chamber and allowed to acclimate for 7-10 days. Plants were treated following the manufacturers recommended application rate of 100 ml of 6× concentrated 16 ml/L SoilBuilder™-AF (Agricen, Pilot Point, Tex., USA). For TI, individual plants were treated with the products only on 1^{st} day, and in parallel with same solutions for 1^{st} day and every 3^{rd} day for TII. Control plants were treated with same water in every 3^{rd} day. Leaves were collected on day 14 (control and treated) and were immediately frozen in liquid nitrogen and stored at −80° C. until RNA extraction.

RNA Extraction, cDNA Synthesis and Quantitative Real-Time PCR (qRT-PCR)

Total RNA was extracted from three biological replicates using TRI-ZOL method following the manufacturer instructions. cDNA synthesis and qPCR analysis was done according to the method of Ali et al. (Ali et al., 2011). Transcript levels in Arabidopsis were measured in triplicates of each biological replicate by qPCR, using SYBR Green (Applied Biosystem) in the Applied BiosystemStepOnePlus™ Real-Time PCR Systems following the manufacturer's manual. The relative mRNA levels were determined by normalizing the PCR threshold cycle number of each gene with that of the

EF-1α (F: CTGGAGGTTTTGAGGCTGGTAT (SEQ ID NO: 105)
and
R: CCAAGGGTGAAAGCAAGAAGA (SEQ ID NO: 106))
and
ACTIN (F: AGCACTTGCACCAAGCAGCATG (SEQ ID NO: 107)
and
R: ACGATTCCTGGACCTGCCTCATC (SEQ ID NO:108)).

as reference genes. The expression level of each gene in the wild-type control or in the sample with the lowest expression level was set to 1, according to GenEx software (http://www.multid.se/order/bioeps.php; GenEx@gene-quantification.info) and the data were the average of three replicates. Sequences of primers used in this study were retrieved from literature and used for amplifying gene-specific sequences (Tables 2 and 3).

### Identification and Quantification of Flavonoids and Anthocyanins by LC-ESI-QTOF-MS/MS Method

Identification and quantification of flavonoids and anthocyanins of Arabidopsis leaves extracts was carried out using an Agilent 1200 LC stack interfaced with an Agilent 6530A (Agilent Technologies, CA, USA) quadrupoletime of flight (Q-TOF) mass spectrometer equipped with an Agilent Jet Stream electrospray ionization (ESI) ion source. The ESI source used a separate nebulizer for the continuous introduction of reference mass compounds: 121.050873, 922.009798 (positive ion mode). Five microlitres of sample extract was separated using an Acquity BEH Shield RP-18 analytical column (1.7 μm 2.1×150 mm, Waters Corporation, Milford, Conn.) maintained at 40° C. The mobile phase of solvent A consists of water/formic acid (99.9:0.1, v/v) and (B) acetonitrile/formic acid (99.9:0.1, v/v) with a solvent ratio of A:B of 95:5. The following gradient for binary pump 1 was used with a total analysis time of 21 min and a flow rate of 0.25 mL/min: 5% to 25% mobile phase B over 2 min then to 25% to 65% mobile phase B for 2.0 to 10.5 min, then to 99% mobile phase B for 10.5 to 12.5 min, then held at 99% mobile phase for 12.5 to 14 min followed by to 5% B from 14 to 15.5 min and then held at 5% 15.5 to 17 min.

The analytical conditions of mass spectrometry are as follows: range, start (100 amu), stop (1,700 amu), and scan time (4.0 s); heating gas temperature, 350° C.; gas flow (l/min), 8.0; nebulizing gas, 35 psi; Sheath gas temp, 350; Sheath gas flow 11.0; VCap 3000; nozzle voltage (V) 1000. The fragmentor voltage was 120 V and skimmer1 65 VandoctopoleRFPeak 750 and collision energies (20V) were optimized for each compound. To confirm the identity of the flavonoids, MS/MS (m/z) fragmentation patterns were compared with those of previously published reports (Tohge et al., 2005) and confirmed by accurate mass QTOF analyses. In the absence of authentic standards, the flavonoids were quantified by peak area. MSMS spectra were compared with LC ESI-Q-TOF-MS/MS spectra of known compounds from the ReSpect data base containing all flavonoids MS/MS spectra (published by Prof Kazuki Saito, JP) and Metlin (the Agilent MS/MS spectral library).

### Statistical Analysis

Statistical analyses of quantitative RT-PCR data were performed by the GenEx software (MultiD analysis) and JPM9 (SAS Institute Inc, Cary, N.C., USA).

### Results

### Metabolite Composition

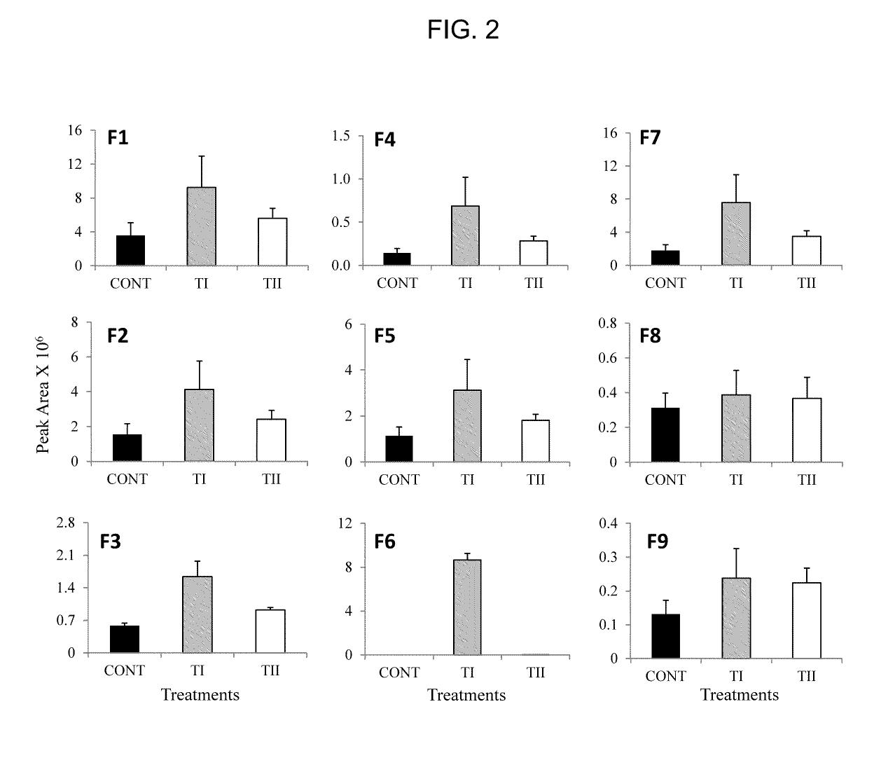

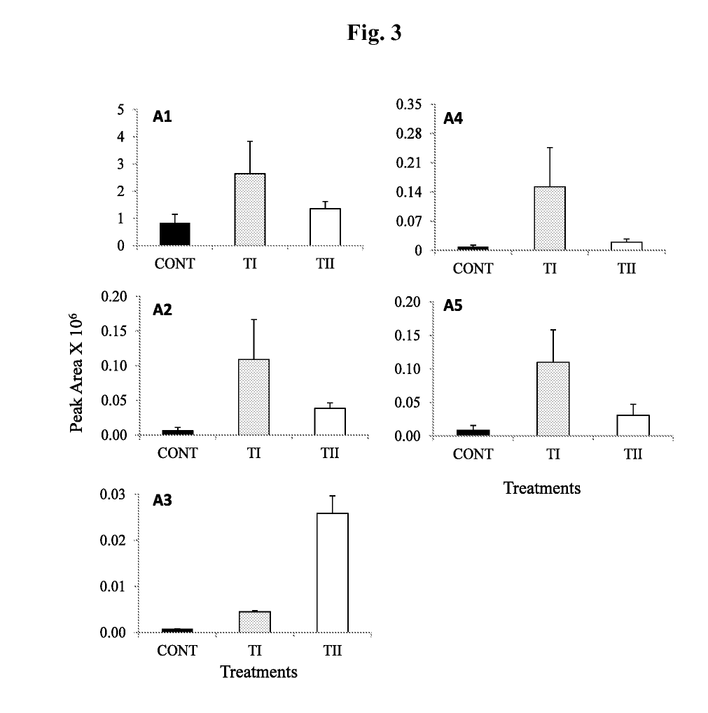

Fourteen flavonoids were identified by HPLC-QTOF-MS/MS analysis in the leaves of Arabidopsis (FIGS. 2 and 3), including nine flavonols (kaempferol-3,7-O-bis-alpha-L-rhamnoside (F1), kaempferol-3-O-alpha-L-rhamnopyranosyl (1-2)-beta-D-glucopyranoside-7-O-alpha-L-rhamnopyranoside (F2), kaempferol with rhamnoside at Rt 7.3 (F3), kaempferol with rhamnoside at Rt 8.2 (F4), kaempferol with rhamnoside at Rt 7.8 (F5), kaempferol in hydrolyzed at Rt 11.4 (F6), quercetin 3,7-dirhamnoside (F7), apigenin7-(2″,3″-diacetylglucoside) (F8) and pentamethoxydihydroxy flavone (F9) and five representative anthocyanidins (cyanidin 3-rhamnoglucoside (A1), (cyanidin 3-(6-malonylglucoside)-7,3′-di-(6-feruloylglucoside) (A2), cyanidin 3-(6″-caffeyl-2″-sinapylsambubioside)-5-(6-malonylglucoside) (A3) and two isomers of cyanidin 3-(2G-glucosylrutinoside) (A4 and A5) (Table 1).

TABLE 1
Flavonoids identified in Arabidopsis thaliana leaf tissue
by liquid chromatography-electrospray ionization Q- time of
flight - mass spectrometry (LC/ESI- Q-TOF MS/MS) analysis.

| Peak | Rt | ESI-MS | Mol | |
|---|---|---|---|---|
| No. | (min) | (m/z) | formula | Compound name |
| 1(A4) | 7.1294 | 757.2185 | C₃₃H₄₁O₂₀ | Anthocyanidin 3-(2G- |
| | | | | glucosylrutinoside) |
| 2(F2) | 7.3303 | 740.2166 | C₃₃H₄₀O₁₉ | Kaempferol-3-O-alpha-L- |
| | | | | rhamnopyranosyl(1-2)-beta- |
| | | | | D-glucopyranoside-7-O- |
| | | | | alpha-L-rhamnopyranoside |
| 3(F3) | 7.3482 | 432.1062 | C₂₁H₂₀O₁₀ | Kaempferol with |
| | | | | rhamnosides |
| 4(A1) | 7.4932 | 595.1674 | C₂₇H₃₁O₁₅ | Anthocyanidin 3- |
| | | | | Rhamnoglucoside |
| 5(A5) | 7.5893 | 757.2188 | | Anthocyanidin 3-(2G- |
| | | | | glucosylrutinoside) |
| 6(F5) | 7.7591 | 432.1062 | C₂₁H₂₀O₁₀ | Kaempferol with |
| | | | | rhamnosides |
| 7(F7) | 7.7687 | 594.1596 | C₂₇H₃₀O₁₅ | Quercetin 3,7-dirhamnoside |
| 8(A2) | 7.7707 | 1211.305 | C₅₆H₅₉O₃₀ | Anthocyanidin 3-(6- |
| | | | | malonylglucoside)-7,3′-di- |
| | | | | (6-feruloylglucoside) |
| 9(A3) | 7.9512 | 1197.293 | C₅₅H₅₇O₃₀ | Anthocyanidin 3-(6″- |
| | | | | caffeyl-2′″- |
| | | | | sinapylsambubioside)- |
| | | | | 5-(6-malonylglucoside) |
| 10(F9) | 8.1942 | 362.0622 | C₁₇H₁₄O₉ | Pentamethoxydihroxy- |
| | | | | flavone |
| 11(F8) | 8.2139 | 516.1278 | C₂₅H₂₄O₁₂ | Apigenin 7-(2″,3″- |
| | | | | diacetylglucoside) |
| 12(F1) | 8.2625 | 578.1646 | C₂₇H₃₀O₁₄ | Kaempferol-3,7-O-bis- |
| | | | | alpha-L-rhamnoside |
| 13(F4) | 8.2657 | 432.1067 | C₂₁H₂₀O₁₀ | Kaempferol with |
| | | | | rhamnosides |
| 14(F6) | 11.434 | 286.048 | C₁₅H₁₀O₆ | Kaempferol |

Rt, retention time; ESI-MS, electrospray ionization/mass spectrometry; m/z, mass/charge;

Significant changes in the concentration of flavonoids occurred, depending on treatment and time of application except F8 (FIGS. 2-3). One time application of products (TI), induce the concentration of F1 (61%), F2 (62%), F3 (64%), F4 (79%), F5 (63%), F6 (99%), F7 (77%), F8 (19%) and F9 (45%) compared to control (FIG. 3). Similarly, but to a lesser extent, concentration of F1 (36%), F2 (35%), F3 (36%), F4 (50%), F5 (37%), F7 (49%), F8 (15%) and F9 (41%) were increased in the TII treatments compared to control (FIG. 3). When compared between TI and TII treatments, TI treatments increase the concentration of F1 (39%), F2 (41%), F3 (43%), F4 (59%), F5 (42%), F6 (99%), F7 (54%), F8 (5%) and F9 (6%) compared to TH. The compound kaempferol, F6 which was detected at Rt11.43 (F6; m/z, 286.04) in the hydrolyzed leafy extracts was induced in the TI treatments compared to control and TII, however, the concentration of kaempferol was very low in control and TII treated plants. Apigenin a flavones containing compound (F8) did not change with the treatments, but pentamethoxydihydroxy flavone (F9) increased in both TI and TII treated plants, while no significant difference was found in the concentration of F9 between the treatments.

The five anthocyanin derivatives (A1-A5) were increased in both TI and TII treated plants compared to control (FIG. 3). One time application of products (TI) induce the concentration of A1 (68%), A2 (94%), A3 (83%), A4 (94%), and A5 (92%) compared to control (FIG. 3). Similarly, TII treatments also increases the concentration of A1 (38%), A2 (84%), A3 (97%), A4 (53%), and A5 (72%) compared to control (FIG. 3). Comparing TI and TII treatments, the TI treatment increased the concentration of A1 (49%), A2 (64%), A4 (87%), and A5 (72%) compared to TII. However, TII treatment increase the concentration of A3 by 82% compared to TI.

### Expression of Flavonoid Biosynthesis Genes in Arabidopsis Leaves

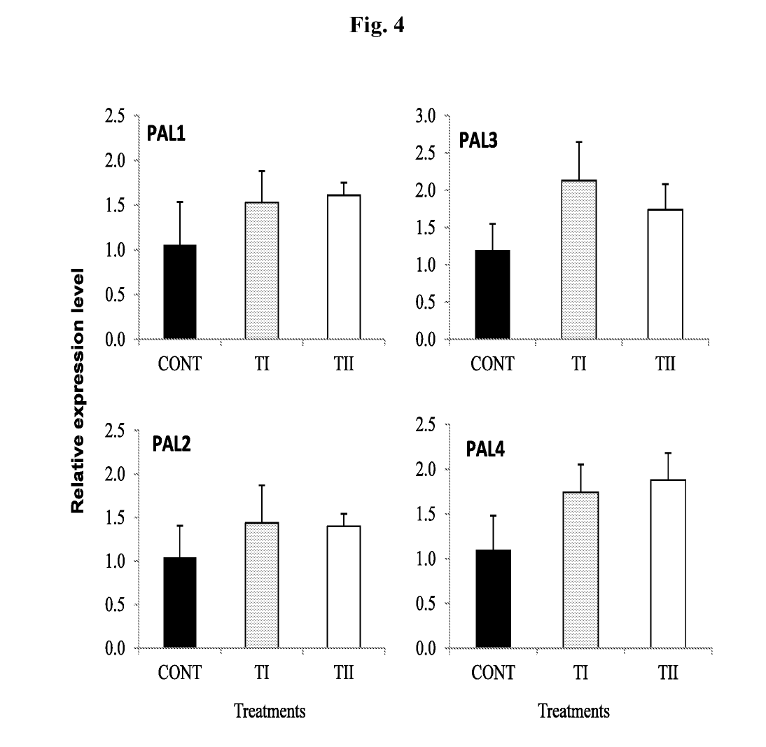

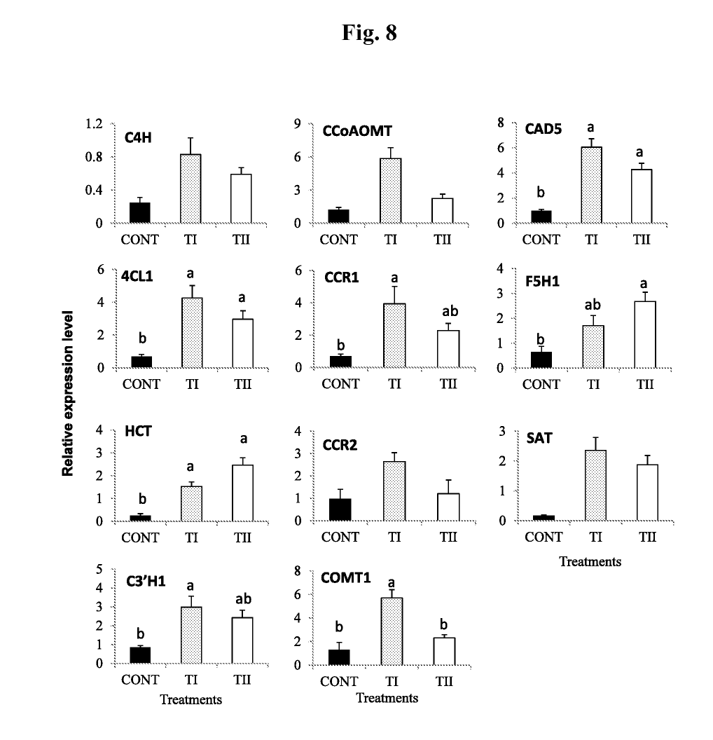

To understand the influence of microbial product application timing (TI and TII) on the flavonoid pathway, the expression of genes encoding key PP pathway enzymes PAL1, PAL2, PAL3, PAL4, C4H, CHS, CH1, F3H, F3′H, FLS1, DFR, LDOX, and UF3GT were analyzed in Arabidopsis leaves using qPCR (FIGS. 4 & 5). Primers used are shown in Table 2. The expression of the upstream genes of the PP pathway (e.g. PALs, C4H and 4CL1) was induced in both types of treatments compared to control. Both types of treatments (TI and TII) induced the expressions of PAL1, PAL2, PAL3 and PAL4 compared to control, while expression of these isoforms was always higher for the treated samples compared to the control (FIG. 4). Expression of C4H and 4CL1 also induced in the TI treatment compared to TII treatment (FIG. 8). The concurrent induction of these genes suggested that the committed steps of upstream PP pathway were induced in both types of treatments.

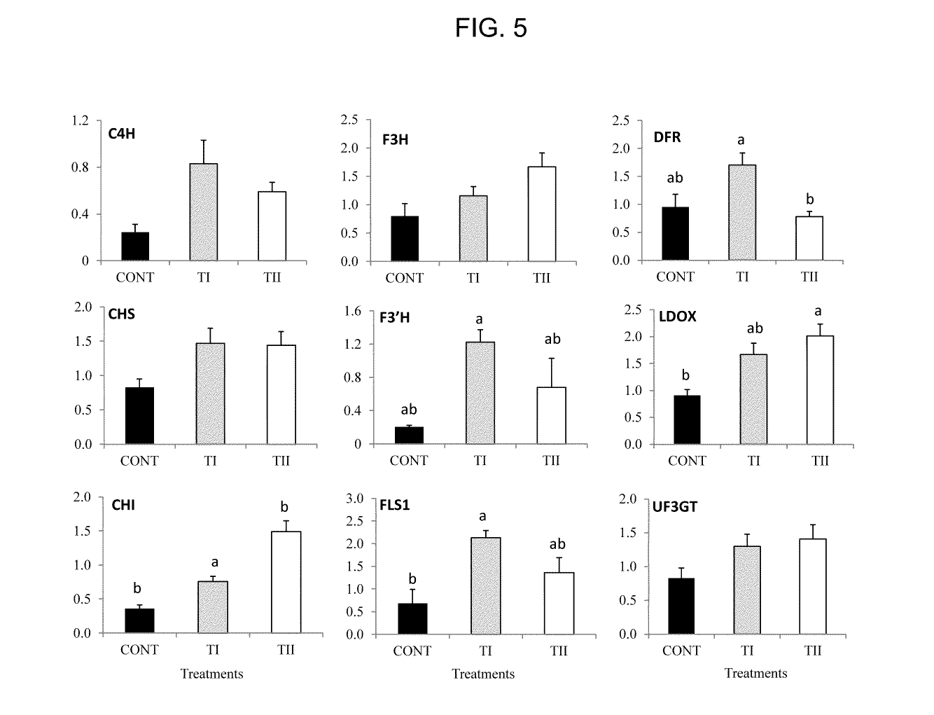

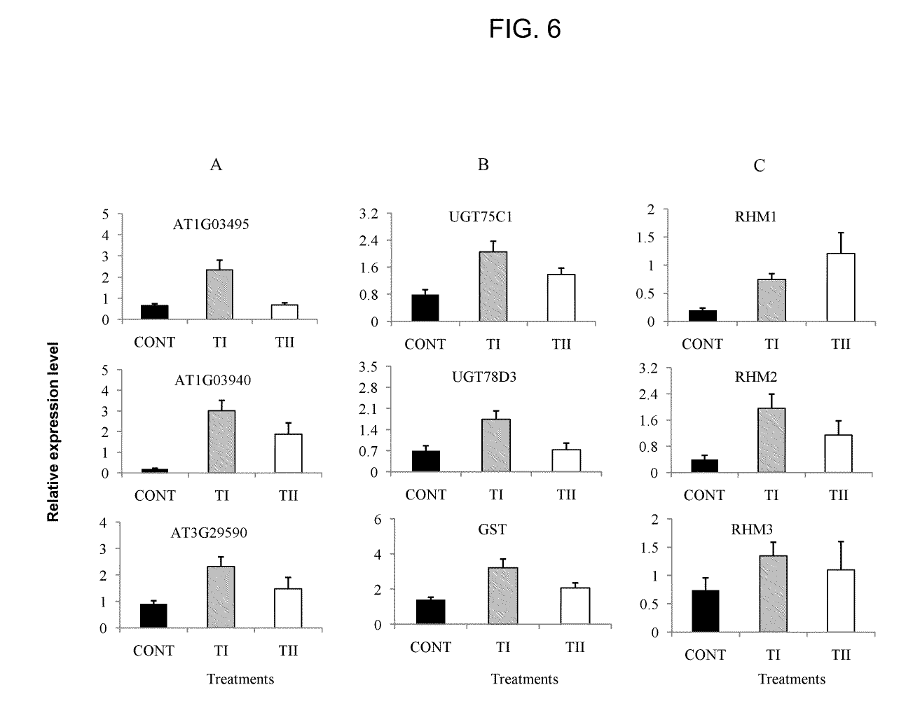

Chalcone synthase (CHS), which marks the beginning of flavonoid biosynthesis, was induced to a similar level in both types of treatments compared to the control (FIG. 5). Similar expression patterns were observed for LDOX and UF3GT. Expression of CHI was also induced relative to the control in TI treated plants. F3′H, FLS1, and DFR followed a similar expression pattern with the only exception being for the TII treatment of DFR which was less than the control. Acylation genes (At1g03495, At1g03940 and At3g29590), glycosylation gene (UGT75C1, and UGT78D3 including GST and UDP-rhamnose synthase genes (RHM1, RHM2, and RHM3) increased in the TI and TII treated plants compared to control (Supplementary FIGS. 6A, 6B and 6C).

### Expression Pattern of Flavonoid Pathway Regulatory Genes in Arabidopsis Leaves

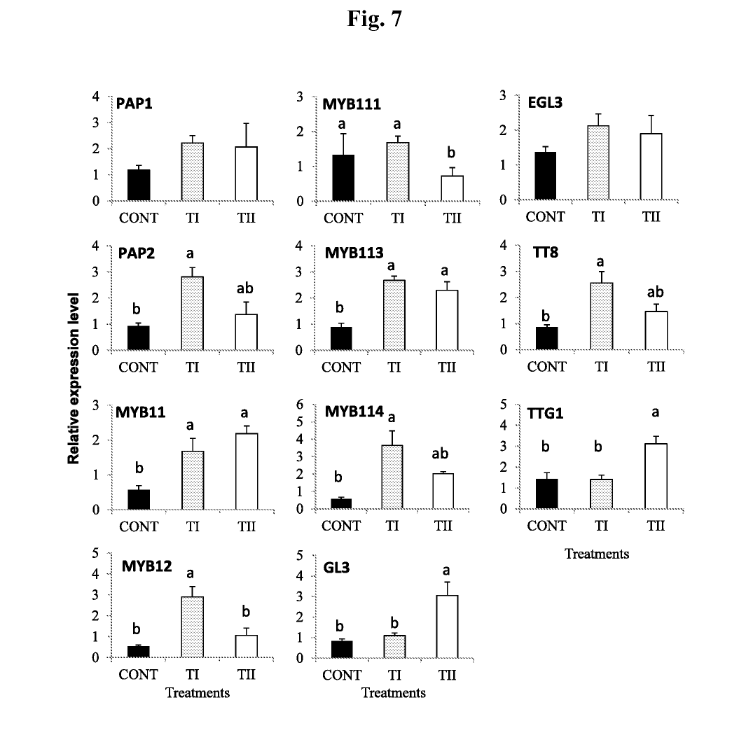

To examine whether the induced expression of flavonoid biosynthetic genes in leaves was accompanied by expression of regulatory genes, the transcript levels of PAP1, PAP2, MYB11, MYB12, MYB111, MYB113, MYB114, GL3, EGL3, TT8 and TTG1 in leaves of Arabidopsis treated with TI and TII (FIG. 7) was analyzed. Expression of most of the regulatory genes was induced both in TI and TII treated plants compared to control. Expression levels of PAP 1 and PAP2 were increased in both TI and TII treated plants compared to the control; more so for PAP2 in the TI treated plants which experienced a 3-fold increase. Expression of MYB11, MYB12, MYB113 and MYB114 were increased in both TI and TII treated plants compared to control. Expression of MYB12 and MYB114 was induced to the greatest extent in the TI compared to TII treated plants. Expression of MYB11 and MYB113 was induced in both TI and TII treated plants compared to control, however, MYB11 expression was higher in TII treated plants compared to TI treated plants. Conversely, MYB11 expression in the TII treatment was suppressed and the TI treatment only slightly up-regulated. The effect of treatment on GL3 and TTG1 expression levels were similar with very little induction for the TI but a clear induction for the TII treated plants. Expression of EGL3 was induced in both TI and TII treated plants compared to control. Strong increase in the expression levels of TT8 and TTG1 were noted in the TI and TII treated plants, respectively compared to control.

TABLE 2
Primers used for quantitative real-time PCR and expected size for the amplified
fragments to Detect Induction of Flavonoid Biosynthesis

| | Sequence of forward (F) and | Seq. ID | Expected | |
|---|---|---|---|---|
| Gene name | reverse (R) primers | No. | size (bp) | Reported by |
| PAL1 | F: GTGTCGCACTTCAGAAGGAA | 1 | 72 | Huang et al. (2010) |
| | R: GGCTTGTTTCTTTCGTGCTT | 2 | | |
| PAL2 | F: GTGCTACTTCTCACCGGAGA | 3 | 77 | Huang et al. (2010) |
| | R: TATTCCGGCGTTCAAAAATC | 4 | | |
| PAL3 | F: CAACCAAACGCAACAGCA | 5 | 78 | Huang et al. (2010) |
| | R: CTCCAGGTGGCTCCCTTTTA | 6 | | |
| PAL4 | F: GGTGCACTTCAAAATGAGCT | 7 | 81 | Huang et al. (2010) |
| | R: CAACGTGTGTGACGTGTCC | 8 | | |
| C4H | F: TGAGTTTGGATCCAGAACGAG | 9 | 115 | In this study |
| | R: CGTCATGATTCTTCTCATCTTCCT | 10 | | |
| CHS | F: CGCATCACCAACAGTGAACAC | 11 | 101 | Kleindt et al. (2010) |
| | R: TCCTCCGTCAGATGCATGTG | 12 | | |
| CHI | F: CCGGTTCATCGATCCTCTTC | 13 | 88 | Kleindt et al. (2010) |
| | R: ATCCCGGTTTCAGGGATACTATC | 14 | | |
| F3H | F: CAGATCGTTGAGGCTTGTGAGA | 15 | 88 | Mehrtens et al. |
| | R: ACGAGTCATATCCGCCACTAAGT | 16 | | (2005) |
| F3′H | F: GCTCTCGCCGGAGTATTCAA | 17 | 74 | Mehrtens et al. |
| | R: CCAGCGACGCCTTGTAAATC | 18 | | (2005) |
| DFR | F: AACGGATGTGACGGTGTTTT | 19 | 93 | Kleindt et al. (2010) |
| | R: TCCATTCACTGTCGGCTTTA | 20 | | |
| LDOX | F: CGATGAAAAGATCCGTGAGAA | 21 | 64 | Mehrtens et al. |
| | R: CACTCCCCAATCCAAAGATG | 22 | | (2005) |
| FLS | F: CCGTCGTCGATCTAAGCGAT | 23 | 107 | Mehrtens et al. |
| | R: CGTCGGAATCCCGTGGT | 24 | | (2005) |
| UDP-GST | F: GATCAGAGGAAGTGATCGAGGA | 25 | 60 | Kleindt et al. (2010) |
| | R: GCCAAAACAGCTGTCTGAGAA | 26 | | |
| UGT78D3 | F: CTCCTCCGATATCCCCACAAA | 27 | 71 | Sakakibara et al. |
| | R: TCAACACGAATCCCTCAGGAA | 28 | | (2008) |
| GST | F: CTTCCGCAACCCTTTTGG | 29 | 154 | In this study |
| | R: GGCTATGCCCGCAATGT | 30 | | |
| UF3GT | F: CAACTGGTTTTCCGTTTCTGGTT | 31 | 64 | Solfanelli et al. |
| | R: GCTTCCTCGACGGTTGATACAC | 32 | | (2006) |
| Acylation | F: CTTCCCTGGAGCTGGAATCT | 33 | 112 | In this study |
| | R: GCCGCTGGATTTGGTCA | 34 | | |
| Acylation | F: CAGAGCCACTTTTACATTGAGC | 35 | | Luo et al., (2007) |
| | R: TCATCCTTGTCTTCCTCGTTG | 36 | | |
| Acylation | F: AGCCACGCTCCTCCACTATC | 37 | 102 | Luo et al., (2007) |
| | R: ACGGCATCTTTGTCGTCAGG | 38 | | |
| RHM1 | F: GGTGGGCGACACTTTGATG | 41 | 78 | Sakakibara et al. |
| | R: CATATGGGTTTGACTTGGTTTTTCA | 42 | | (2008) |
| RHM2 | F: TCTACAATGTCGGCACAAAAAGA | 43 | 73 | Sakakibara et al. |
| | R: TTCCCGAAAAGTTTGCAGATG | 44 | | (2008) |
| RHM3 | F: ATGCAGATGGTAATCAGACATTCAC | 45 | 78 | Sakakibara et al. |
| | R: AGGATCTTTTGTCTCCGGAACA | 46 | | (2008) |
| PAP1 | F: AAATGGCACCAAGTTCCTGT | 47 | 113 | In this study |
| | R: TCAGAGCTAAGTTTTCCTCTCTTGAT | 48 | | |
| PAP2 | F: GACTGCTGAAGAAGATAGTCTCTTG | 49 | 104 | Velten et al. (2010) |
| | R: GCCCAGCTCTCAAAGGAACTTGATG | 50 | | |
| MYB11 | F: GGCGATTGTAACCCAAGCATT | 51 | 116 | Gou et al. |
| | R: TCACATGAGGACACGTGGACA | 52 | | (2011) |
| MYB12 | F: TGATGGGGAGTTGCATAACATA | 53 | 114 | In this study |
| | R: AACGACTCCACCGATGGAC | 54 | | |
| MYB111 | F: AATAACAAGACCAAGAAGAAGAAGAA | 55 | 92 | In this study |
| | R: AGAAACATTGTGAGGCCGTC | 56 | | |
| MYB113 | F: ATCTTGTTCTTCGCCTTCATAAA | 57 | 134 | Gou et al. |
| | R: GCATCGTTCATCGTGCTTCTTA | 58 | | (2011) |
| MYB114 | F: GTCTCTTGAGGCAGTGTATTGGT | 59 | 87 | Qi et al. |
| | R: TTTTCCTGCACCGATTTAGC | 60 | | (2011) |
| GL3 | F: AGTGTTTAGCCGTTCTCTTCTAGC | 61 | 113 | Kleindt et al. (2010) |
| | R: TGTCTTCCGTAATATGTTCTGTGG | 62 | | |
| EGL3 | F: TTGGCACGACCGAACATA | 63 | 100 | Kleindt et al. (2010) |
| | R: TTGATAGTCTGATCTTGTCGATATTGT | 64 | | |
| TT8 | F: TGAATCAACCCATACGTTAGACA | 65 | 102 | Kleindt et al. (2010) |
| | R: GGGGTGTGACATGAGAAGTGT | 66 | | |
| TTG1 | F: TCCTCGAAGATTACAACAACCG | 67 | 72 | In this study |
| | R: CGGGAGAGGCTTAACGGTCAT | 68 | | |

### Lignin Biosynthesis

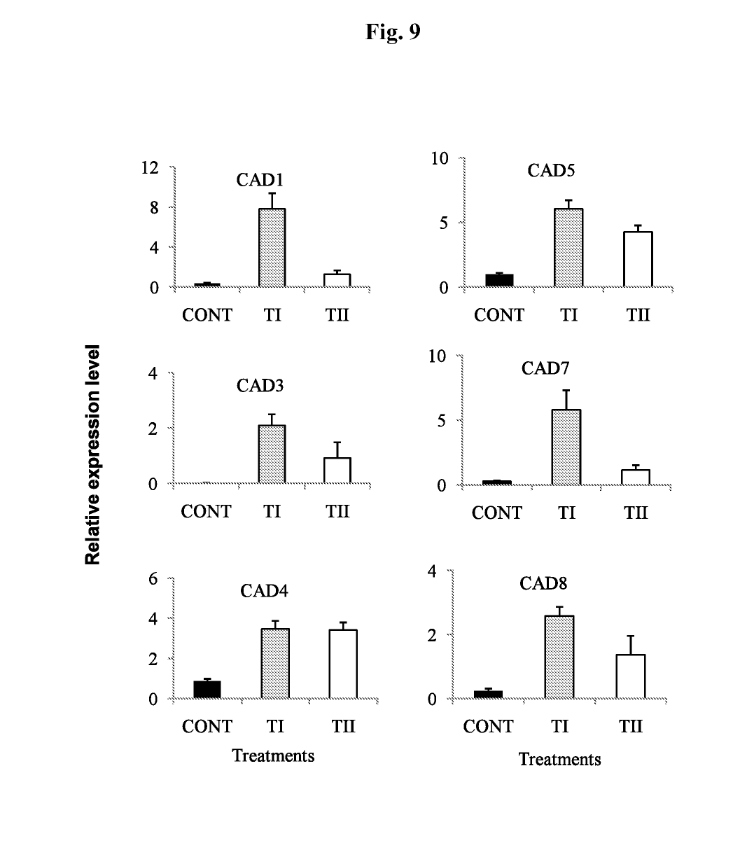

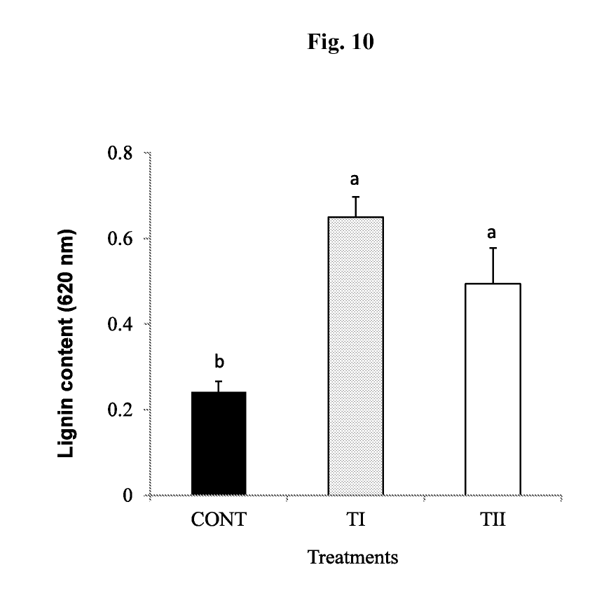

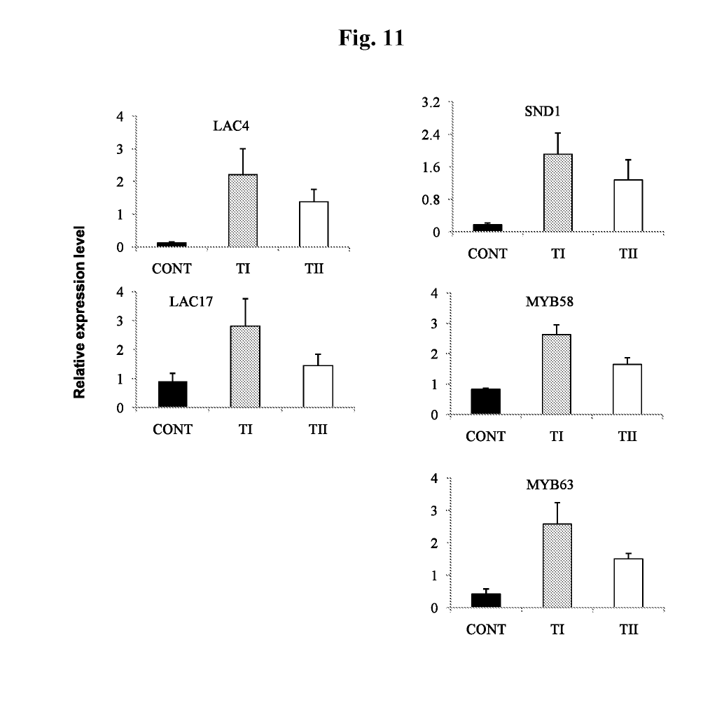

To further understand the application of microbial treatments (TI and TII), we analyzed the expression of all the genes (20) involved in the lignification pathway (FIG. 8 and FIG. 9). Primers used are shown in Table 3. The accumulation of transcripts for the four PAL isoforms, C4H and 4CL1, were induced in both treatments; more so for TI than TII. These results indicate that TI and TII treatments induce the expression of the three genes of the general phenylpropanoid pathway which is in line with greater total lignin content in treated plants (FIG. 10). The expression patterns of C3′H1, CCoAOMT, CCR1, CCR2, COMT1, CAD1, CAD3, CAD5, CAD7, CAD8 and SAT were all increased several fold in the TI treated plants compared to TII treated plants. In contrast, expression of HCT, and CAD4 was increased in the TII treated plants compared to TI treated plants. Expression of LAC4 and LAC17 and their regulatory genes (SND1, MYB58, and MYB63) showed greater expression in both TI and TII treated plants compared to control (FIG. 11) with greater expression levels for the TI compared to the TII treated plants.

TABLE 3
Primers used for quantitative real-time PCR and expected size for the amplified
fragments to Detect Induction of Lignin Biosynthesis

| | Sequence of forward (F) and | Seq. ID | Expected | |
|---|---|---|---|---|
| Gene name | reverse (R) primers | No. | size (bp) | Reported by |
| SAT | F: CTGCTGCTATAGTCAAGTCTCTTCC | 39 | | In this study |
| | R: GAGAGAAGAACATCCAGGTCCTC | 40 | | |
| 4CL1 | F: TCAACCCGGTGAGATTTGTA | 69 | 132 | Bhargava et al. |
| | R: TCGTCATCGATCAATCCAAT | 70 | | (2010) |
| HCT | F: GCCTGCACCAAGTATGAAGA | 71 | 136 | Bhargava et al. |
| | R: GACAGTGTTCCCATCCTCCT | 72 | | (2010) |
| C3′H | F: GTTGGACTTGACCGGATCTT | 73 | 104 | Bhargava et al. |
| | R: ATTAGAGGCGTTGGAGGATG | 74 | | (2010) |
| CCoAOMT | F: CTCAGGGAAGTGACAGCAAA | 75 | 146 | Bhargava et al. |
| | R: GTGGCGAGAAGAGAGTAGCC | 76 | | (2010) |
| CCR1 | F: GTGCAAAGCAGATCTTCAGG | 77 | 153 | Bhargava et al. |
| | R: GCCGCAGCATTAATTACAAA | 78 | | (2010) |
| F5H | F: CTTCAACGTAGCGGATTTCA | 79 | 87 | Bhargava et al. |
| | R: AGATCATTACGGGCCTTCAC | 80 | | (2010) |
| COMT1 | F: TTCCATTGCTGCTCTTTGTC | 81 | 199 | Bhargava et al. |
| | R: CATGGTGATTGTGGAATGGT | 82 | | (2010) |
| CAD3 | F: ATCGTTTCGGATATTGAGCTCATAAA | 83 | 133 | Sibout et al. (2005) |
| | R: TCTCAGCTGACGACTCAGGGAGTAAA | 84 | | |
| CAD4 | F: TCTGGTGGAGGAGGCTGCAACA | 85 | 144 | Hossain et al., |
| | R: AGCCAAAGCATTCGTGTTTGAACCA | 86 | | (2011) |
| CAD5 | F: TTGGCTGATTCGTTGGATTA | 87 | 164 | Bhargava et al. |
| | R: ATCACTTTCCTCCCAAGCAT | 88 | | (2010) |
| CAD7 | F: CGATGAAGCCAACTCCTTAACTAGAAA | 89 | 141 | Sibout et al. (2005) |
| | R: CAACTGATAAAGTACATGCAGTG | 90 | | |
| | TGGTAATA | | | |
| CAD8 | F: CATTGAAGCCTAATCCTAATTTATA | 91 | 144 | Sibout et al. (2005) |
| | AGTTTTAA | | | |
| | R: ACTATTCATTTATTGGATTAAGCAT | 92 | | |
| | ACCAAATTA | | | |
| CAD9 | F: CCTCCATGAATGATCCGGATCTAA | 93 | 153 | Sibout et al. (2005) |
| | GAA | | | |
| | R: CGAAATAAGGTAACTTGTCTGAGA | 94 | | |
| | AGAAA | | | |
| LAC4 | F: GGTGGATGGGTCGTCATGAGATTC | 95 | 213 | Sawa et al. |
| | R: CGTGGCGTGATGTTGATATGTCGCCC | 96 | | (2005) |
| LAC17 | F: GGCCATTTATCGGTTTGACAT | 97 | 100 | Minic et al. (2009) |
| | R: CAGAAAACAGAGCTGTGCAAC | 98 | | |
| SND1 | F: CCCGAGTTCGCTTTCCAGGTG | 99 | 141 | Hossain et al. (2011) |
| | R: CAGGACGAACCGGGCAACAGT | 100 | | |
| MYB58 | F: CCAGAGAACAGAGCTCTTCAAGAG | 101 | 180 | Zhou et al. (2008) |
| | R: ATGTATGAGGAGCTCGTAACTCTC | 102 | | |
| MYB63 | F: GAACAGCTCAGGCTCAAGAGCAAC | 103 | 178 | Zhou et al. (2008) |
| | R: ATGTATCATGAGCTCGTAGTTCTT | 104 | | |

### CONCLUSIONS

This study shows that microbial products applied to the soil of growing plants results in induction of the PP pathway and increased secondary metabolite biosynthesis. The one time application of microbial products (TI) produced more metabolites than multiple applications (TII). However, overall flavonoid accumulation was higher in the treated plants, regardless of timing, compared to the control. Such differences in the flavonols and anthocyanin accumulations between TI and TII treated plants can be explained by the differential transcript accumulation of structural and regulatory genes in leaves of Arabidopsis.

This invention may be embodied in other forms or carried out in other ways without departing from the spirit or essential characteristics thereof. The present disclosure is therefore to be considered as in all aspects illustrate and not restrictive, and all changes which come within the meaning and range of equivalency are intended to be embraced therein.

Various references are cited throughout this specification, each of which is incorporated herein by reference in its entirety.

### REFERENCES

Adesemoye A O, Torbert H A, Kloepper J W. “Plant growth-promoting Rhizobacteria allow reduced application rates of chemical fertilizers” Microbial Ecology 58, 921-929. 2009.
Ali M, Howard S, Chen S, Wang Y, Yu O, Kovacs L, Qiu W. “Berry skin development in Norton grape: Distinct patterns of transcriptional regulation and flavonoid biosynthesis” Bmc Plant Biology 11, 7. 2011.
Barea J M, Pozo M J, Azcon R, Azcon-Aguilar C. “Microbial co-operation in the rhizosphere” Journal of Experimental Botany 56, 1761-1778. 2005.
Bhargava A, Mansfield S D, Hall H C, Douglas C J, Ellis B E. “MYB75 functions in regulation of secondary cell wall formation in the Arabidopsis inflorescence stem” Plant Physiology 154, 1428-1438. 2010.
Burkett-Cadena M, Kokalis-Burelle N, Lawrence K S, van Santen E, Kloepper J W. “Suppressiveness of root-knot nematodes mediated by rhizobacteria” Biological Control 47, 55-59. 2008.
DeFries R, Rosenzweig C. “Toward a whole-landscape approach for sustainable land use in the tropics.” Proceedings of the National Academy of Sciences 107, 19627-19632. 2010.
Dodd I C, Perez-Alfocea F. “Microbial amelioration of crop salinity stress” Journal of Experimental Botany 63, 3415-3428. 2012.
García-Mediavilla V, Crespo I, Collado P S, Esteller A, Sánchez-Campos S, Tuñón M J, González-Gallego J. “The anti-inflammatory flavones quercetin and kaempferol cause inhibition of inducible nitric oxide synthase, cyclooxygenase-2 and reactive C-protein, and down-regulation of the nuclear factor kappaB pathway” in Chang Liver cells. European Journal of Pharmacology 557, 221-229. 2007.
Green R E, Cornell S J, Scharlemann J P W, Balmford A. “Farming and the fate of wild nature” Science 307, 550-555. 2005.
Hossain Z, Amyot L, McGarvey B, Gruber M, Jung J, Hannoufa A. “The translation elongation factor eEF-1Bβ1 is involved in cell wall biosynthesis and plant development in Arabidopsis thaliana” PLOS One 7, e30425. 2012.
Huang J, Gu M, Lai Z, Fan B, Shi K, Zhou Y-H, Yu J-Q, Chen Z. “Functional analysis of the Arabidopsis PAL gene family in plant growth, development, and response to environmental stress”, Plant Physiol. 153, 1526-1538. 2010.
Kleindt C K, Stracke R, Mehrtens F, Weisshaar B. “Expression analysis of flavonoid biosynthesis genes during Arabidopsis thaliana silique and seed development with a primary focus on the proanthocyanidin biosynthetic pathway” BMC research notes 3, 255. 2010.
Koh E, Charoenprasert S, Mitchell A., “Effect of organic and conventional cropping systems on ascorbic acid, vitamin C, flavonoids, nitrate, and oxalate in 27 varieties of spinach (Spinacia oleracea L.)” J Agr Food Chem 60, 3144-3150. 2012.
Kutchan T M. “A role for intra- and intercellular translocation in natural product biosynthesis” Current Opinion in Plant Biology 8, 292-300. 2005.
Luo J, Nishiyama Y, Fuell C, Taguchi G, Elliott K, Hill L, Tanaka Y, Kitayama M, Yamazaki M, Bailey P et al. “Convergent evolution in the BAHD family of acyl transferases: identification and characterization of anthocyanin acyl transferases from Arabidopsis thaliana” The Plant Journal 50, 678-695. 2007.
Mehrtens F, Kranz H, Bednarek P, Weisshaar B. “The Arabidopsis transcription factor MYB12 is a flavonol-specific-regular of Phenylpropanoid Biosynthesis” Plant Physiol 138, 1083-1096. 2005.
Minic Z, Jamet E, San-Clemente H, Pelletier S, Renou J-P, Rihouey C, Okinyo D, Proux C, Lerouge P, Jouanin L. “Transcriptomic analysis of Arabidopsis developing stems: a close-up on cell wall genes” BMC Plant Biol 9, 6. 2009.
Morgan J A, Bending G D, White P J. “Biological costs and benefits to plant-microbe interactions in the rhizosphere” Journal of Experimental Botany 56, 1729-1739. 2005.
Morrissey J P, Dow J M, Mark G L, O'Gara F. “Are microbes at the root of a solution to world food production?” EMBO Rep 5, 922-926. 200.4
Onekura-Sakakibara K, Tohge T, Matsuda F, Nakabayashi R, Takayama H, Niida R, Watanabe-Takahashi A, Inoue E, Saito K. “Comprehensive flavonol profiling and transcriptome coexpression analysis leading to decoding gene-metabolite correlations in Arabidopsis” The Plant Cell Online 20, 2160-2176. 2008.
Quentin M, Allasia V, Pegard A, Allais F, Cucrot P-H, Favery B, Levis C, Marinet S, Masur C, Ponchet M, et al. “Imbalanced lignin biosynthesis promotes the sexual reproduction of homothallic Oomycete pathogens” PLOS Pathogens 5, e1000264. 2009.
Ruiz-López N, Sayanova O, Napier J A, Haslam R P. “Metabolic engineering of the omega-3 long chain polyunsaturated fatty acid biosynthetic pathway into transgenic plants” Journal of Experimental Botany 63, 2397-2410. 2012.
Saber M S M. “Clean biotechnology for sustainable farming” Chemical Engineering & Technology 24, A217-A223. 2001.
Sawa S, Demura T, Horiguchi G, Kubo M, Fukuda H. “The ATE genes are responsible for repression of transdifferentiation into xylem cells in Arabidopsis” Plant Physiol 137, 141-148. 2005.
Sibout R, Eudes A, Mouille G, Pollet B, Lapierre C, Jouanin L, Seguin A. “CINNAMYL ALCOHOL DEHYDROGENASE-C and -D are the primary genes involved in lignin biosynthesis in the floral stem of Arabidopsis” Plant Cell 17, 2059-2076. 2005.
Solfanelli C, Poggi A, Loreti E, Alpi A, Perata P. “Sucrose-specific induction of the anthocyanin biosynthetic pathway in Arabidopsis” Plant Physiol 140, 637-646. 2006.
Stracke R, Ishihara H, Barsch G H A, Mehrtens F, Niehaus K, Weisshaar B. “Differential regulation of closely related R2R3-MYB transcription factors controls flavonol accumulation in different parts of the Arabidopsis thaliana seedling” Plant Journal 50, 660-677. 2007.
Sung P H, Huang F C, Do Y Y, Huang P L. “Functional expression of geraniol 10-hydroxylase reveals its dual function in the biosynthesis of terpenoid and phenylpropanoid” Journal of Agricultural and Food Chemistry 59, 4637-4643. 2011.
Taie H, El-Mergawi R, Radwan S. “Isoflavonoids, flavonoids, phenolic acids and antioxidant activity of soybean seeds as affected by organic and bioorganic fertilization” American-Eurasian J. Agric. Environ. Sci. 4, 207-213. 2008.
Tohge T, Nishiyama Y, Hirai M Y, Yano M, Nakajima J, Awazuhara M, Inoue E, Takahashi H, Goodenowe D B, Kitayama M, Noji M, Yamazaki M, Saito K. “Functional genomics by integrated analysis of metabolome and transcriptome of Arabidopsis plants over-expressing an MYB transcription factor” Plant Journal 42, 218-235. 2005.
Vandermeer J, Perfecto I. “The Future of farming and conservation” Science 308, 1257-1258. 2005.
Vanholme R, Demedts B, Morreel K, Ralph J, Boerjan W. “Lignin biosynthesis and structure” Plant Physiology 153, 895-905. 2010.
Velten J, Cakir C, Youn E, Chen J, Cazzonelli C I. “Transgene silencing and transgene-derived siRNA production in tobacco plants homozygous for an introduced AtMYB90 construct” PLOS One 7, e30141. 2012.
Ververidis F, Trantas E, Douglas C, Vollmer G, Kretzschmar G, Panopoulos N. “Biotechnology of flavonoids and other phenylpropanoid-derived natural products. Part I: Chemical diversity, impacts on plant biology and human health” Biotechnology Journal 2, 1214-1234. 2007.
Wang H, Nair M, Strasburg G, Chang Y-C, Booren A, Gray J, SeWitt D. “Antioxidant and antiinflammatory activities of anthocyanins and their aglycon, cyanidin, from tart cherries” J. Nat. Prod. 62, 294-296. 1999.
Whipps J M. “Microbial interactions and biocontrol in the rhizosphere” Journal of Experimental Botany 52, 487-511. 2001.
Winkel-Shirley B. “Flavonoid biosynthesis. A colorful model for genetics, biochemistry, cell biology, and biotechnology” Plant Physiology 126, 485-493. 2001.
Worthington V. “Nutritional quality of organic versus conventional fruits, vegetables, and grains” J Alt Compl Med 7, 161-173, 2001.
Yildirim E, Taylor A G, Spittler T D. “Ameliorative effects of biological treatments on growth of squash plants under salt stress” Scientia Horticulturae 111, 1-6. 2006.
Zhou J L, Lee C H, Zhong R Q, Ye Z H. “MYB58 and MYB63 are transcriptional activators of the lignin biosynthetic pathway during secondary cell wall formation in Arabidopsis” Plant Cell 21, 248-266. 2009.

What is claimed is:

1. A method for inducing, stimulating or otherwise promoting and/or modulating at least one of: (a) flavonoid biosynthesis; (b) lignin biosynthesis in a plant in need thereof comprising applying to soil and/or said plant an amount of a microbial based soil additive effective to induce and/or modulate flavonoid and/or lignin biosynthesis.

2. The method according to claim 1, wherein said flavonoid biosynthesis induced and/or modulated in said plant is at least one of flavone biosynthesis, flavonol, dihydroflavanol and/or anthocyanadin biosynthesis.

3. The method according to claim 1, wherein said flavonoid biosynthesis induced is biosynthesis of at least one of a kaempferol or derivative thereof, an apigenin or derivative thereof, a quercitin or derivative thereof, a dihydroxyflavone and/or cyanadin derivative.

4. The method according to claim 3, wherein said kaempferol derivative, cyanidin derivative and/or quercetin derivative is a glycosylated derivative.

5. The method according to claim 3, wherein said cyanidin derivative is an acylated derivative.

6. The method according to claim 1, wherein said flavonoid biosynthesis induced biosynthesis of at least one of Anthocyanidin 3-(2G-glucosylrutinoside), Kaempferol-3-O-alpha-L-rhamnopyranosyl(1-2)-beta-D-glucopyranoside-7-O-alpha-L-rhamnopyranoside, Kaempferol with rhamnosides, Anthocyanidin 3-Rhamnoglucoside, Anthocyanidin 3-(2G-glucosylrutinoside), Kaempferol with rhamnosides, Quercetin 3,7-dirhamnoside, Anthocyanidin 3-(6-malonylglucoside)-7,3′-di-(6-feruloylglucoside), Anthocyanidin 3-(6″-caffeyl-2′″-sinapylsambubioside)-5-(6-malonylglucoside), Pentamethoxydihroxyflavone, Apigenin 7-(2″,3″-diacetylglucoside), Kaempferol-3,7-O-bis-alpha-L-rhamnoside, Kaempferol.

7. A method for inducing, stimulating or otherwise promoting and/or modulating expression of (a) one or more structural genes and/or (b) one or more glycosylation genes, and/or (c) one or more acylation genes and/or (d) one or more regulatory genes of the phenylpropanoid pathway in a plant in need thereof comprising applying to soil and/or said plant an amount of a soil additive derived from a bioreactor effective to induce, stimulate or otherwise promote and/or modulate expression of structural genes, and/or regulatory genes of the phenylpropanoid pathway.

8. The method according to claim 7, wherein expression of said structural gene induced is at least one of (a) one or more isoforms of PAL, (b) F3H, (c) F3′H, (d) FLS1, (e) DFR, (f) LDOX, (g) C4H; (h) 4CL1, (i) CHS, (j) CH1 and/or (k) UF3GT.

9. The method according to claim 7, wherein expression of said structural gene induced is PAL1, PAL2, PAL3 and/or PAL4.

10. The method according to claim 7, wherein glycosylation is induced and/or modulated by expression of UDP glycosyl transferase gene and/or glutathione S transferase gene.

11. The method according to claim 7, wherein glycosylation is induced and/or modulated by expression of UGT75C1, UGT78D3, UDP rhamnose synthase gene and/or GST.

12. The method according to claim 11, wherein said UDP rhamnose synthase gene is RHM1, RHM2 and RHM3.

13. The method according to claim 7, wherein expression of said acylation gene induced and/or modulated is At1g03495, At1g03940 and/or At3g29590 gene.

14. The method according to claim 7, wherein expression of said regulatory gene induced is one or more of a MYB and/or PAP transcription factor.

15. The method according to claim 7, wherein expression of said transcription factor induced is a MYB11, MYB12, MY113, MYB114, GL3, EGL3, TT8 and/or TTG1.

16. The method according to claim 7, wherein expression of said transcription factor induced is a PAP1 and/or PAP2.

17. A method of inducing, stimulating or otherwise promoting and/or modulating lignin biosynthesis in a plant in need thereof comprising applying an amount of a microbial based soil additive effective to induce and/or modulate lignin biosynthesis, wherein said lignin biosynthesis is induced and/or modulated by at least one of expression of (a) one or more lignin biosynthesis genes; (b) one or more laccase genes and/or (c) one or more or more lignin regulatory genes.

18. The method according to claim 17 wherein said lignin biosynthesis genes are selected from the group consisting of 4CL1, HCT, C3′H1, CCoAOMT, CCR1, CCR2, COMT1, CAD1, CAD3, CAD5, CAD7, CAD8 and SAT.

19. The method according to claim 18, wherein said laccase genes are selected from the group consisting of LAC4 and LAC17.

20. The method according to claim 18, wherein said lignin regulatory genes are selected from the group consisting of MYB63, SND1 and MYB58.

21. The method according to 1, wherein said soil additive is derived from SoilBuilder™-AF (SB) (Agricen, Pilot Point, Tex.) or filter-sterilized broth of SoilBuilder™-AF (FSB) (Agricen, Pilot Point, Tex.).

22. The method according to claim 1, wherein said soil additive is a composition comprising a composition that has the following characteristics: (a) has a pH of about 7.5 to 8.8; (b) COD range less than about 150 mg/L; (c) Conductivity range of about 1200 uS to 3800 uS; (d) Color clear amber between about 500 pt/co units to about 700 pt/co units in a platinum to cobalt (pt/co) scale; (e) comprises at least one of Syntrophus, Desulfovibrio, Symbiobacteria, Georgfuschia, Thauera, Nitrosomonas, Bellilinea, Sulfuritalea, and Owenweeksia; (f) has a culturable plate count of greater than 10⁶ microbes per ml.; (g) contains between about 10-60 ng/ml DNA; (h) comprises at least eight microbial species or filter-sterilized broth thereof.

23. A method for detecting induction of flavonoid biosynthesis in a plant in need thereof by a microbial based soil additive comprising:
(a) providing a sample from a plant and
(b) contacting said sample with one or more probe or primers having at least about 97% homology to a nucleotide sequence wherein said nucleotide sequence is SEQ ID NOS:1-38 and 41-68.

24. The method according to claim 23, wherein the method further comprises comparing the level of flavonoid biosynthesis to the level of flavonoid biosynthesis in a sample from a plant not treated.

25. A method for detecting induction of lignin biosynthesis in a plant in need thereof by a microbial based soil additive comprising:
(a) providing a sample from a plant and
(b) contacting said sample with one or more probe or primers having at least about 97% homology to a nucleotide sequence wherein said nucleotide sequence is SEQ ID NOS:39-40 and 69-104.

26. The method according to claim 25, wherein the method further comprises comparing the level of lignin biosynthesis to the level of flavonoid biosynthesis in a sample from a plant not treated.

27. An oligonucleotide probe or primer for detecting flavonoid and/or lignin biosynthesis in a plant having a sequence of at least 17 nucleotides with at least about 97% homology to a nucleotide sequence, wherein said sequence is selected from the group consisting of SEQ ID NOS: 9, 10, 33, 34, 39, 40, 47, 48, 53, 54, 55, 56, 67, 68.

28. A kit for detecting flavonoid and/or lignin biosynthesis in a plant comprising SEQ ID NOS: 1-104.
